(19) United States
(12) Patent Application Publication
(10) Pub. No.: US 2026/0216168 A1
(43) Pub. Date: Jul. 30, 2026

(54) MODULATION OF PD-1 SIGNALING FOR TREATMENT OF DISEASES

(71) Applicant: Wuhan University, Wuhan, Hubei (CN)

(72) Inventors: Hongbing Shu, Wuhan, Hubei (CN); Shu Li, Wuhan, Hubei (CN); Rui Liu, Wuhan, Hubei (CN)

(21) Appl. No.: 19/159,136

(22) PCT Filed: Sep. 22, 2023
(86) PCT No.: PCT/CN2023/120563

§ 371 Date: Aug. 22, 2025

(30) Foreign Application Priority Data
Feb. 27, 2023 (WO) PCT/CN2023/078365

(51) Int. Cl. A61K 31/47; A61K 38/20; A61K 38/48; A61K 39/00; A61P 35/00; C07K 16/28; C07K 16/40; C12N 15/113

(52) U.S. Cl. CPC A61K 31/47; A61K 38/2013; A61K 38/4813; A61P 35/00; C07K 16/2818; C07K 16/2827; C07K 16/40; C12N 15/1137; A61K 2039/505; C12N 2310/11; C12N 2310/141; C12N 2310/531; C12Y 203/02; C12Y 301/03048

(57) ABSTRACT

Provided are compositions and methods for treating cancer, infection or Parkinson's disease. An example treatment entails administration of an agent that inhibits the biological activity or expression of MARCH5 (membrane-associated ring finger 5) or SIIP2 (src homology region 2 domain-containing phosphatase-2) or increases the biological activity or expression of USP5 (ubiquitin specific peptidase 5), optionally in combination with a common gamma-chain cytokine and/or a PD-1 inhibitor or a PD-L1 inhibitor.

### BACKGROUND

Programmed cell death protein 1 (PD-1, also referred to as CD279) is a key coinhibitory receptor in the B7-CD28-CTLA-4 family which is widely expressed in lymphocytes. PD-L1 (B7-H1 or CD274) and PD-L2 (B7-DC or CD273) are the ligands for PD-1 which are mostly expressed in various immune cells. Mammals evolve coinhibitory pathways to control the magnitude and duration of T cell responses to limit tissue damage and maintain self-tolerance under physiological and pathological conditions. However, tumor cells hijack these inhibitory pathways to escape host immune surveillance by overexpression of PD-L1. This provides the rationale for clinical application of immune checkpoint inhibitors in cancer immunotherapy. Recent studies have also revealed that PD-1 signal regulates the functions ofNK cells in tumor microenvironment (TME).

Clinically, antibodies blocking the PD-1/PD-L1 axis reinvigorate the exhausted T cells in TME and show remarkable objective response and durable remission with acceptable toxicity profile in large numbers of tumors such as lymphoma and melanoma. However, not all patients respond to PD-1 monotherapy and there is considerable interest in developing combination therapy to improve the overall response rate and trigger more complete and durable response in patients with cancer.

Common cytokine receptor γ chain (γ_{c}, also referred to as CD132) is a component of the receptors for interleukin-2 (IL-2), IL-4, IL-7, IL-9, IL-15 and IL-21. γ_{c} is widely expressed in immune cells, and mutation of the gene encoding γ_{c} (IL2RG) results in X-linked severe combined immunodeficiency. Cytokines of the γ_{c} family exhibit pleiotropic functions in both innate and adaptive immune responses, contribute to development of T, B, NK and innate lymphoid cells (ILCs), promote either survival or death of immune cells depending on the context, and modulate differentiation of precursor immune cells into more terminally differentiated cells. Because of their important roles in regulating activity of T, NK and other immune cells, some of the γ_{c} family cytokines, such as IL-2, IL-9, IL15 and IL21, have shown strong anti-tumor effects. Studies of the γ_{c} family cytokines have allowed remarkable translational advances for autoimmune diseases as well as cancer.

In the past years, combination immunotherapy of cancer has shown great promises. PD-1 blockade plus the γ_{c} family cytokine IL-2 is a promising combination of cancer immunotherapy with several clinical trials ongoing. The utility of IL-2 as a therapeutic agent for cancer is recognized early after its discovery, based on its powerful ability to stimulate proliferation of cytotoxic T lymphocytes and NK cells. However, only high-dose of IL-2 has shown therapeutic effects in certain cancer patients, and widespread utilization is also limited by systemic toxicity, whereas combination therapy with PD-1 blockade and IL-2 is highly effective in cancer patients. Understanding the underlying mechanisms responsible for the synergistic effects of this combination is important to design better strategies for cancer immunotherapy.

### SUMMARY

Combination therapy with PD-1 blockade and IL-2 substantially improves anti-tumor efficacy comparing to monotherapy. The underlying mechanisms responsible for the synergistic effects of combination therapy remain unknown. It has been discovered herein that PD-1 ligation results in BATF-dependent transcriptional induction of the membrane-associated E3 ubiquitin ligase MARCH5, which mediates K27-linked polyubiquitination and lysosomal degradation of the common cytokine receptor γ chain (γ_{c}). PD-1 ligation also activates SHP2, which dephosphorylates γ_{c}^{Y357}, leading to impairment of γ_{c} family cytokine-triggered signaling. Conversely, PD-1 blockade restores γ_{c} level and activity, thereby sensitizing CD8⁺ T and NK cells to IL-2.

MARCH5 inhibition, when combined with PD-1 blockade and IL-2, significantly improved the efficacy of anti-tumor immunotherapies. These findings uncover the mechanisms on how PD-1 signal antagonizes γ_{c} family cytokine-triggered immune activation and demonstrate that the underlying mechanisms can be exploited for increased efficacy of combination immunotherapy of cancer. Also, these treatments can also be effectively used for treating infection.

Accordingly, compositions and methods are provided for treating cancer. An example treatment entails administration of an agent that inhibits the biological activity or expression of MARCH5 (membrane-associated ring finger 5) or SHP2 (src homology region 2 domain-containing phosphatase-2) or increases the biological activity or expression of USP5 (ubiquitin specific peptidase 5), optionally in combination with a common gamma-chain cytokine and/or a PD-1 inhibitor or a PD-L1 inhibitor.

One embodiment of the present disclosure provides a method for treating cancer or infection in a patient in need thereof, comprising administering to the patient an effective amount of an agent that inhibits the biological activity or expression of MARCH5 (membrane-associated ring finger 5) or SHP2 (src homology region 2 domain-containing phosphatase-2) or increases the biological activity or expression of USP5 (ubiquitin specific peptidase 5).

In some embodiments, the method further comprises administering to the patient a common gamma-chain cytokine, or wherein the patient has received or is prescribed to receive a therapy comprising a common gamma-chain cytokine. In some embodiments, the common gamma-chain cytokine is selected from the group consisting of IL-2, IL-4, IL-7, IL-9, IL-15, IL-21, and combinations thereof. In some embodiments, the common gamma-chain cytokine comprises IL-2.

In some embodiments, the method further comprises administering to the patient a PD-1 inhibitor or a PD-L1 inhibitor, or wherein the patient has received or is prescribed to receive a therapy comprising a PD-1 inhibitor or a PD-L1 inhibitor. In some embodiments, the PD-1 or PD-L1 inhibitor is an anti-PD-1 or anti-PD-L1 antibody of antigen-binding fragment thereof. In some embodiments, the PD-1 inhibitor is selected from the group consisting of pembrolizumab, nivolumab, cemiplimab, spartalizumab, camrelizumab, sintilimab, tislelizumab, dostarlimab, INCMGA00012, AMP-224 and AMP-514.

In some embodiments, the PD-L1 inhibitor is selected from the group consisting of atezolizumab, avelumab, durvalumab, KN035, CK-301, AUNP12, CA-170, BMS-986189, B6 and B12-01. In some embodiments, the anti-PD-1 or anti-PD-L1 antibody is a fusion protein that further comprises a common gamma-chain cytokine.

In some embodiments, the agent is a small molecule MARCH5 inhibitor. In some embodiments, the small molecule MARCH5 inhibitor is selected from the group consisting of pitavastatin, lonafarnib, tucidinostat, belinostat, mocetinostat, pracinostat, risedronate, entinostat, chidamide, vorinostat, and salts thereof. In some embodiments, the agent is pitavastatin calcium.

In some embodiments, the agent is an antibody or antigen-binding thereof that targets MARCH5 or SHP2. In some embodiments, the agent is an inhibitory RNA that targets MARCH5 or SHP2. In some embodiments, the inhibitory RNA is a shRNA, siRNA, miRNA, piRNA, or antisense RNA. In some embodiments, the agent is a recombinant USP5 protein or polynucleotide encoding the USP5 protein.

In some embodiments, the cancer is selected from the group consisting of bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, sarcoma, skin cancer, squamous cell carcinoma, bone cancer, melanoma, renal cell carcinoma, and kidney cancer. In some embodiments, the cancer is a cold tumor.

In some embodiments, the infection is a bacterial infection or viral infection.

Also provided, in one embodiment, is a method for preventing or treating Parkinson's disease in a patient in need thereof, comprising administering to the patient an effective amount of an agent that inhibits the biological activity or expression of MARCH5 (membrane-associated ring finger 5). In some embodiments, the agent is selected from the group consisting of pitavastatin, lonafarnib, tucidinostat, belinostat, mocetinostat, pracinostat, risedronate, entinostat, chidamide, vorinostat, and salts thereof.

Also provided is a kit or package comprising (a) an agent that inhibits the biological activity or expression of MARCH5 (membrane-associated ring finger 5) or SHP2 (src homology region 2 domain-containing phosphatase-2) or increases the biological activity or expression of USP5 (ubiquitin specific peptidase 5), and (b1) a common gamma-chain cytokine and/or (b2) a PD-1 inhibitor or a PD-L1 inhibitor.

### BRIEF DESCRIPTION OF THE DRAWINGS

FIG. 1 shows that PD-1 signal is negatively correlated with γ_{c} level.
(A) γ_{c} level is negatively correlated with PD-L1 level in human NSCLC tumor biopsies. Representative images from IHC staining of γ_{c} and PD-L1 in human NSCLC tumor biopsies are shown (left panels). Quantification of PD-L1 and γ_{c} staining intensities were performed by semi-quantitative scoring (right panels). n=127 independent samples, R=−0.2966. Correlation coefficients were calculated using the Pearson test. Two-sided P-value was given. Scale bar, 100 μm.
(B) PD-1 blockade increases γ_{c} level in tumor tissues. B16F10 cells (5×10⁵) were subcutaneously injected into C57BL/6j mice. CT26 cells (5×10⁵) were subcutaneously injected into Balb/c mice. Mice were intraperitoneally injected with control or anti-PD-1 antibody (100 μg per mouse) every three days (four times in total) five days after inoculation of cells. After 17 days, tumor-bearing mice were euthanized and tumor tissues were analyzed. Representative images from IHC staining of γ_{c} in tumor sections are shown. Scale bar, 100 μm.
(C) PD-1 blockade increases γ_{c} level in tumor-infiltrating CD8⁺ T and NK cells. TILs were isolated from B16F10 tumor tissues (left panels) or CT26 tumor tissues (right panels), stained with the indicated antibodies and analyzed by flow cytometry. Graph shows mean±SEM, n=8 independent samples. Data were analyzed using a student's unpaired t-test with GraphPad Prism 8. MFI, median fluorescence intensities.
(D) PD-1 ligation down-regulates γ_{c} level. Human CD8⁺ T cells or PD-1-expressing Jurkat cells were stimulated with anti-CD3/CD28 (1 μg/ml of anti-CD3, 5 μg/ml of anti-CD28) in the presence of 2 g/ml human PD-L1-Fc fusion protein or control (human IgG1) for 3 days. The cells were stained with the indicated antibodies and analyzed by flow cytometry. Graph shows mean±SEM, n=3 technical repeats. Data were analyzed using two-way ANOVA with GraphPad Prism 8.
(E) The level of γ_{c} is down-regulated after PD-1 ligation. Human CD8⁺ T cells or PD-1-expressing Jurkat cells were stimulated with anti-CD3/CD28 (1 μg/ml of anti-CD3, 5 μg/ml of anti-CD28) in the presence of 2 μg/ml human PD-L1-Fc fusion protein or control (human IgG1) for 3 days before immunoblotting analysis with the indicated antibodies. PD-1-expressing Jurkat cells were stimulated with PHA (50 ng/ml) in the presence of 2 μg/ml human PD-L1-Fc fusion protein or control (human IgG1) for the indicated times before immunoblotting analysis with the indicated antibodies.

FIG. 2 shows that MARCH5 mediates K27-linked polyubiquitination and lysosomal degradation of yc.
(A) PD-1 ligation promotes γ_{c} degradation. PD-1-expressing Jurkat cells were pre-stimulated with PHA (50 ng/ml) for 36 h and then treated with CHX (0.1 mM) for the indicated times before immunoblotting analysis with the indicated antibodies. The γ_{c} band intensities relative to the corresponding β-actin bands were shown in the histograph.
(B) NH₄Cl inhibits γ_{c} degradation. Jurkat cells were pre-treated with MG132 (100 μM), NH₄Cl (25 mM) or 3-MA (500 ng/ml) for 4 h, and then treated with CHX (0.1 mM) for 2 h before immunoblotting analysis with the indicated antibodies.
(C) NH₄Cl inhibits PD-1 ligation-induced degradation of γ_{c}. PD-1-expressing Jurkat cells were stimulated with PHA (50 ng/ml) in the presence of 2 μg/ml human PD-L1-Fc fusion protein or control (human IgG1) for 36 h, and then treated with MG132 (100 PM), NH₄Cl (25 mM) or 3-MA (500 ng/ml) for 12 h before immunoblotting analysis with the indicated antibodies.
(D) PD-1 ligation induces polyubiquitination of γ_{c}. PD-1-expressing Jurkat cells were stimulated with PHA (50 ng/ml) for the indicated times before co-immunoprecipitation and immunoblotting analysis with the indicated antibodies.
(E) MARCH5 promotes degradation of γ_{c} in a dose-dependent manner. HEK293 cells were transfected with the indicated plasmids for 24 h before immunoblot analysis with the indicated antibodies.
(F) Association of γ_{c} with MARCH5. PD1-expressing Jurkat cells were stimulated with PHA (50 ng/ml) in the presence of 2 μg/ml human PD-L1-Fe fusion protein or control (human IgG1) for the indicated times before co-immunoprecipitation and immunoblotting analysis with the indicated antibodies.
(G) MARCH5-deficiency up-regulates the level of γ_{c} but not IL2RP. Control or MARCH5-deficient Jurkat cells were collected for immunoblotting analysis with the indicated antibodies.
(H) MARCH5-deficiency impairs PD-1 ligation-induced degradation of γ_{c}. Control or MARCH5-deficient PD-1-expressing Jurkat cell were stimulated with PHA (50 ng/ml) in the presence of 2 μg/ml human PD-L1-Fc fusion protein or control (human IgG1) for the indicated times before immunoblotting analysis with the indicated antibodies.
(I) MARCH5 promotes polyubiquitination of γ_{c}. HEK293 cells were transfected with the indicated plasmids for 24 h before co-immunoprecipitation and immunoblotting analysis with the indicated antibodies.
(J) MARCH5-deficiency impairs PD-L1-induced K27-linked polyubiquitination of γ_{c}. Control or MARCH5-deficient PD-1-expressing Jurkat cells were stimulated with PHA (50 ng/ml) in the presence of 2 μg/ml human PD-L1-Fc fusion protein or control (human IgG1) for the indicated times before co-immunoprecipitation and immunoblotting analysis with the indicated antibodies.
(K) MARCH5 increases K27-linked polyubiquitination of wild-type γ_{c} and γ_{c}^{K294R} but not γ_{c}^{K315R} HEK293 cells were transfected with the indicated plasmids for 24 h before co-immunoprecipitation and immunoblotting analysis with the indicated antibodies.
(L) PD-1 induces down-regulation of wild-type γ_{c} but not γ_{c}^{K315R}. PD-1-expressing Jurkat cells were expressed with Flag-tagged wild-type γ_{c} but not γc^{K315R} mutant and then stimulated with PHA (50 ng/ml) in the presence of 2 μg/ml human PD-L1-Fc fusion protein or control (human IgG1) for the indicated times before immunoblotting analysis with the indicated antibodies.

FIG. 3 shows that USP5 removes K27-linked polyubiquitin moieties from γ_{c}.
(A) USP5 removes K27-linked polyubiquitin moieties from γ_{c}. HEK293 cells were transfected with the indicated plasmids for 24 h before immunoblotting analysis with the indicated antibodies.
(B) Association of γ_{c} with USP5. PD-1-expressing Jurkat cells were stimulated with PHA (50 ng/ml) in the presence of 2 μg/ml human PD-L1-Fc fusion protein or control (human IgG1) for the indicated times before co-immunoprecipitation and immunoblotting analysis with the indicated antibodies.
(C) USP5 but not its enzymatic inactive mutant USP5^{C335A} removes K27-linked polyubiquitin moieties from γ_{c}. HEK293 cells were transfected with the indicated plasmids for 24 h before co-immunoprecipitation and immunoblotting analysis with the indicated antibodies.
(D) USP5 removes K27-linked polyubiquitin moieties of γ_{c} catalyzed by MARCH5. HEK293 cells were transfected with the indicated plasmids for 24 h before immunoblotting analysis with the indicated antibodies.
(E) USP5-deficiency enhances PD-1 ligation-induced K27-linked polyubiquitination of γ_{c}. Control or USP5-deficient PD-1-expressing Jurkat cells were stimulated with PHA (50 ng/ml) in the presence of 2 μg/ml human PD-L1-Fc fusion protein or control (human IgG1) for the indicated times before co-immunoprecipitation and immunoblotting analysis with the indicated antibodies.

FIG. 4 shows that PD-1 signal promotes γ_{c} degradation by inducing transcription of MARCH5.
(A) MARCH5 is up-regulated after PD-L1 but not PHA stimulation. Human CD8⁺ T cells or PD-1-expressing Jurkat cells were stimulated with anti-CD3/CD28 (1 μg/ml of anti-CD3, 5 μg/ml of anti-CD28) in the presence of 2 μg/ml human PD-L1-Fc fusion protein or control (human IgG1) for 3 days before immunoblotting analysis with the indicated antibodies (left panels). PD-1-expressing Jurkat cells were stimulated with PHA (50 ng/ml) in the presence of 2 μg/ml human PD-L1-Fc fusion protein or control (human IgG1) for the indicated times before immunoblotting analysis with the indicated antibodies (right panels).
(B) Effects of PD-1 ligation on MARCH5 degradation. PD-1-expressing Jurkat cells were pre-stimulated with PHA (50 ng/ml) in the presence of 2 μg/ml human PD-L1-Fc fusion protein or control (human IgG1) for 36 h and then treated with CHX (0.1 mM) for the indicated times before immunoblotting analysis with the indicated antibodies. The MARCH5 band intensities relative to the corresponding R-actin bands were shown in the histograph.
(C) MARCH5-deficiency impairs PD-1 ligation-induced degradation of γ_{c}. Control or MARCH5-deficient PD-1-expressing Jurkat cells were reconstituted with wild-type MARCH5 or MARCH5^{H43W} mutant and then stimulated with PHA (50 ng/ml) in the presence of 2 μg/ml human PD-L1-Fc fusion protein or control (human IgG1) for 2 days before immunoblotting analysis with the indicated antibodies.
(D) Effects of PD-1 ligation on MARCH5 mRNA level. Human CD8⁺ T cells or PD-1-expressing Jurkat cells were stimulated with anti-CD3/CD28 (1 μg/ml of anti-CD3, 5 μg/ml of anti-CD28) in the presence of 2 μg/ml human PD-L1-Fe fusion protein or control (human IgG1) for 3 days before qPCR analysis of mRNA levels of the indicated genes. Graph shows mean±SEM, n=3 independent samples from one representative experiment. Data were analyzed using two-way ANOVA with GraphPad Prism 8.
(E) BATF binds to the promoter region of MARCH5 gene. PD-1-expressing Jurkat cells were analyzed by ChIP with the indicated antibodies, and then de-crosslinked DNA was subjected to qPCR analysis using specific primers. Graph shows mean±SEM, n=3 independent samples from one representative experiment. Data were analyzed using a student's unpaired t-test with GraphPad Prism 8.
(F) BATF-deficiency impairs PD-1 ligation-induced transcription of MARCH5. Control or BATF-deficient PD-1-expressing Jurkat cells were stimulated with PHA (50 ng/ml) in the presence of 2 μg/ml human PD-L1-Fc fusion protein or control (human IgG1) for 2 days before qPCR analysis of mRNA levels of the indicated genes. Graph shows mean±SEM, n=3 independent samples from one representative experiment. Data were analyzed using two-way ANOVA with GraphPad Prism 8.
(G) BATF-deficiency impairs PD-1 ligation-induced degradation of γ_{c}. Control or BATF-deficient PD-1-expressing Jurkat cells were stimulated with PHA (50 ng/ml) in the presence of 2 μg/ml human PD-L1-Fc fusion protein or control (human IgG1) for 2 days before immunoblotting analysis with the indicated antibodies.

FIG. 5 shows PD-1 signals activation of SHP2 to dephosphating γ_{c}^{Y357}.
(A) Association of γ_{c} with SHP2. PD-1-expressing Jurkat cells were stimulated with PHA (150 ng/ml) in the presence of 2 μg/ml human PD-L1-Fe fusion protein or control (human IgG1) for the indicated times before co-immunoprecipitation and immunoblotting analysis with the indicated antibodies.
(B) Effects of SHP2 on dephosphorylation of γ_{c}. HEK293 cells were transfected with the indicated plasmids for 24 h before co-immunoprecipitation and immunoblotting analysis with the indicated antibodies.
(C) JAK3 mediates phosphorylation of γ_{c}^{Y357}. HEK293 cells were transfected with the indicated plasmids for 24 h before co-immunoprecipitation and immunoblotting analysis with the indicated antibodies.
(D) IL-7 treatment induces γ_{c}^{Y357} phosphorylation. Control or γ_{c}-deficient HPB-ALL cells were stimulated with IL-7 (100 ng/ml) for the indicated times before co-immunoprecipitation and immunoblotting analysis with the indicated antibodies.
(E) SHP2-deficiency impairs PD-L1-induced γ_{c}^{Y357} dephosphorylation. Control or SHP2-deficient PD-1-expressing Jurkat cells were stimulated with PHA (150 ng/ml) in the presence of 2 μg/ml human PD-L1-Fc fusion protein or control (human IgG1) for the indicated times before co-immunoprecipitation and immunoblotting analysis with the indicated antibodies.
(F) SHP2-deficiency enhances IL-7-induced γ_{c}^{Y357} phosphorylation. Control or SHP2-deficient HPB-ALL cells were stimulated with IL-7 (100 ng/ml) for the indicated times before co-immunoprecipitation and immunoblotting analysis with the indicated antibodies.
(G) SHP2-deficiency promotes γ_{c} family cytokine-induced phosphorylation of STAT5^{Y694/Y699}. Control or SHP2-deficient HPB-ALL cells were stimulated with IL-9 (100 ng/ml) for the indicated times before immunoblotting analysis with the indicated antibodies (upper panels). Control or SHP2-deficient CTLL2 cells were stimulated with IL-2 (400 IU/ml) for the indicated times before immunoblotting analysis with the indicated antibodies (lower panels).
(H) Effects of γ_{c}^{Y357F} mutant on the γ_{c} family cytokine-induced phosphorylation of STAT5^{Y694/Y699}. γ_{c}-deficient HPB-ALL cells were reconstituted with wild-type γ_{c} or γ_{c}^{Y357F} mutant and then stimulated with IL-7 (100 ng/ml) or IL-9 (100 ng/ml) for the indicated times before immunoblotting analysis with the indicated antibodies.

FIG. 6 shows that MARCH5 knockdown improves anti-tumor immunity and suppresses tumor growth.
(A) Effects of MARCH5 knockdown on the level of γ_{c} in lymphocytes from spleen. Splenocytes from sex- and age-matched March5^{+/f} or March5^{+/f:Vav1-Cre} mice were stained with the indicated antibodies and analyzed by flow cytometry. Graph shows mean±SEM, n=6 independent samples. Data were analyzed using a student's unpaired t-test with GraphPad Prism 8. MFI, median fluorescence intensities.
(B) March5 knockdown increases the percentage of CD8⁺ single-positive (CD8SP) cells in thymocytes. Thymocytes from sex- and age-matched March5^{+/f} or March5^{+/f:Vav1-Cre} mice were analyzed by flow cytometry for the percentage of CD4⁻CD8⁻ double-negative (DN), CD4⁺CD8⁺ double-positive (DP), CD4⁺ single-positive (CD4SP) and CD8⁺ single-positive (CD8SP). The CD4⁻CD8⁻ double-negative (DN) cells were analyzed by flow cytometry for the percentage of CD44⁺ single-positive (DN1), CD44⁺CD25⁺ double-positive (DN2), CD25⁺ single-positive (DN3) and CD44⁻CD25⁻ double-negative (DN4). Graph shows mean±SEM, n=6 independent samples. Data were analyzed using a student's unpaired t-test with GraphPad Prism 8.
(C) Effects of MARCH5 knockdown on the percentages of CD8⁺ T and NK cells from spleen and the peripheral blood. Splenocytes or peripheral blood leukocytes from sex- and age-matched March5^{+/f} or March5^{+/f:Vav1-Cre} mice were analyzed by flow cytometry for the percentage of CD8⁺ T (CD3⁺CD8⁺), NK (CD3⁻NKp46⁺), native CD8⁺ T (CD44^{low}CD62L^{high} CD8⁺ T cells), central memory CD8⁺ T (CD44^{high}CD62L^{high} CD8⁺ T cells, CM) and effector/effector memory CD8⁺ T (CD44^{high}CD62L^{low}CD8⁺ T cells, Effector/EM) cells. Graph shows mean±SEM, n=6 independent samples. Data were analyzed using a student's unpaired t-test with GraphPad Prism 8.
(D) MARCH5 knockdown inhibits tumor growth. Sex- and age-matched March5^{+/f} or March5^{+/f:Vav1-Cre} mice were subcutaneously injected with MC38 cells (5×10⁵). On day 3 after tumor cell inoculation, tumor sizes were measured every two days by caliper. Tumor-bearing mice were euthanized on day 13, and then tumor tissues were separated from the mice. Tumor weights were measured by Analytical Balance. Graph shows mean±SEM, n=6. Data were analyzed using a student's unpaired t-test with GraphPad Prism 8.

(E) MARCH5 knockdown increases the percentages of CD8⁺ T and NK cells in TILs. TILs were isolated from the MC38 tumor tissues in FIG. 6D. TILs were stained with the indicated antibodies and analyzed by flow cytometry. Graph shows mean±SEM, n=6 independent samples. Data were analyzed using a student's unpaired t-test with GraphPad Prism 8.

(F) Combination of IL-2 and PD-1 blockade has increased anti-tumor efficacy in March5^{+/f:Vav1-Cre} mice. March5^{+/f} and March5^{+/f:Vav1-Cre} mice were subcutaneously injected with MC38 cells (5×10⁵). On day 5 after tumor cell inoculation, mice were intraperitoneally injected with control, IL-2 (50000 IU per mouse) or anti-PD-1 (100 μg per mouse). Tumor sizes were measured every two days by caliper from day 5. WT: March5^{+/f}, CHZ: March5^{+/f:Vav1-Cre}. Graph shows mean±SEM, n=8. Data were analyzed using two-way ANOVA with GraphPad Prism 8.

(G) Combination of IL-2 and PD-1 blockade increases the survival rate in March5^{+/f:Vav1-Cre} mice. March5^{+/f} and March5^{+/f:Vav1-Cre} mice were subcutaneously injected with MC38 cells (5×10⁵). On day 5 after tumor cell inoculation, mice were intraperitoneally injected with control, IL-2 (50000 IU per mouse) or anti-PD-1 (100 μg per mouse). Mice were sacrificed when the tumor size is bigger than 15 mm of the mean tumor diameter, tumor volume exceeded 2000 mm³, or tumor had ulcers with diameter reached 10 mm. Statistical analysis was performed using the GraphPad Prism 8 software, n=8. Kaplan-Meier survival curves and corresponding log-rank (Mantel-Cox) tests were used to evaluate the statistical differences between groups in survival studies. There is a significant difference when the P<0.05.

FIG. 7 shows that pitavastatin calcium potentiates anti-tumor immunity triggered by combination therapy of IL-2 and PD-1 blockade.
(A) PC treatment up-regulates the level of γ_{c}. Human CD8⁺ T, mouse CD8⁺ T, Jurkat, HBP-ALL or CTLL2 cells were treated with PC (0, 0.5, 1, 2 μM) for 24 h before immunoblotting analysis with the indicated antibodies.
(B) MARCH5-deficiency impairs PC-induced up-regulation of γ_{c}. Control or MARCH5-deficient Jurkat cells were treated with PC (0, 0.5, 1 μM) for 24 h before immunoblotting analysis with the indicated antibodies.
(C) PC treatment suppresses tumor growth. C57BL/6j mice were subcutaneously injected with 5×10⁵ of MC38 or B16F10 cells. On day 3 (MC38) or 5 (B16F10) after tumor cell implantation, mice were intraperitoneally injected with control or PC (5 mg/kg/day). Tumor sizes were measured every two days by caliper. Tumor-bearing mice were euthanized on day 13 (MC38) or day 15 (B16F10). Tumor weights were measured by Analytical Balance. Graph shows mean±SEM, n=6. Data were analyzed using a student's unpaired t-test with GraphPad Prism 8.
(D) Effects of PC on the level of γ_{c} in TILs. TILs were isolated from the MC38 tumor tissues of FIG. 7C. TILs were stained with the indicated antibodies and analyzed by flow cytometry. Graph shows mean±SEM, n=6 independent samples. Data were analyzed using a student's unpaired t-test with GraphPad Prism 8. MFI, median fluorescence intensities.
(E) PC treatment increases tumor infiltrating CD8⁺ cytotoxic T cells. TILs were isolated from the MC38 tumor tissues of FIG. 7C. TILs were stained with the indicated antibodies and analyzed by flow cytometry. Graph shows mean±SEM, n=6 independent samples. Data were analyzed using a student's unpaired t-test with GraphPad Prism 8.
(F) PC potentiates the anti-tumor efficacy of IL-2 and PD-1 combination. C57BL/6j mice were subcutaneously injected with MC38 cells (5×10⁵). On day 5 after tumor cell implantation, mice were intraperitoneally injected with control, PC (5 mg/kg), IL-2 (50000 IU per mouse) or anti-PD-1 (100 μg per mouse). Tumor sizes were measured every two days by caliper from day 5. Graph shows mean±SEM, n=8. Data were analyzed using two-way ANOVA with GraphPad Prism 8.
(G) PC promotes the survival rate of mice treated with IL-2 and PD-1 blockade. C57BL/6j mice were subcutaneously injected with MC38 cells (5×10⁵). On day 5 after tumor cell implantation, mice were intraperitoneally injected with control, PC (5 mg/kg), IL-2 (50000 IU per mouse) or anti-PD-1 (100 μg per mouse). Mice were sacrificed when the tumor size is bigger than 15 mm of the mean tumor diameter, tumor volume exceeded 2000 mm³, or tumor had ulcers with diameter reached 10 mm. Statistical analysis was performed using the GraphPad Prism 8 software, n=8. Kaplan-Meier survival curves and corresponding log-rank (Mantel-Cox) tests were used to evaluate the statistical differences between groups in survival studies. There is a significant difference when the P<0.05.

FIG. 8 presents a model on regulation of γ_{c} stability and activity by PD-1 signal. In tumor microenvironment, PD-L1/PD-1 signaling results in inhibition of the γ_{c} family cytokine-triggered signaling and immune activation by two mechanisms. Immediately after PD-1 ligation, SHP2 is recruited to PD-1 and activated, which in turn dephosphorylating γ_{c} at Y357, leading to its inactivation and unresponsiveness to γ_{c} family cytokines. More later after PD-1 ligation, the transcription factor BATF is induced, which induces expression of the membrane-associated E3 ubiquitin ligase MARCH5. MARCH5 is recruited to γ_{c} and mediates its K27-linked polyubiquitination at K315 and lysosomal degradation. Targeting of components involved in these regulatory mechanisms, such as by a combination of PD-1 blocking antibody (1), IL-2 (2) and MARCH5 inhibitor (3) leads to potent anti-tumor effects.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ 1-γ4). It is the nature of this chain that determines the “class” of the antibody as IgG, IgM, IgA IgG, or IgE, respectively. The immunoglobulin subclasses (isotypes) e.g., IgG1, IgG₂, IgG₃, IgG4, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgAQ1 and IgA2) or subclass of immunoglobulin molecule.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Modulation of PD-1 Pathway for Treatment of Cancer

Combination therapy with PD-1 blockade and IL-2 substantially improves anti-tumor efficacy compared with PD-1 blockade or IL-2 monotherapy, which has been extensively explored for immunotherapy of various cancers. The basic principle of combined immunotherapy with PD-1 blockade and IL-2 is to remove the PD-1 inhibitory brake and in the meantime provide a stimulatory signal for cytotoxic T lymphocytes and NK cells with IL-2. Recent studies have also shown that combination immunotherapy with PD-1 blockade and IL-2 modifies CD8⁺ T cell exhaustion program. Currently, the cellular and molecular basis responsible for the synergistic effects observed between PD-1 blockade and IL-2 remains unknown.

As discovered by the instant inventors in the experimental examples, PD-1 signal negatively regulates the stability and activity of γ_{c}, which impairs the γ_{c} family cytokine-triggered signaling and immune activation of CD8⁺ T and NK cells in the TME. PD-1 blockade removes the inhibitory effects on γ_{c}, thereby restoring the responses of CD8⁺ T and NK cells to the γ_{c} family cytokines such as IL-2 and leading to the synergistic effects of combination immunotherapy of PD-1 blockade and IL-2. Moreover, the data revealed two distinct molecular mechanisms responsible for PD-1 signal-triggered inhibition of γ_{c}-mediated immune activation, and validated the membrane-associated E3 ligase MARCH5 as a potential target for combination immunotherapy.

The data showed that overexpression of MARCH5 but not its inactive mutant MARCH5^{H43W} promoted K27-linked polyubiquitination and lysosomal degradation of γ_{c}. MARCH5-deficiency up-regulated the level of γ_{c}, which was reversed by reconstitution with wild-type MARCH5 but not MARCH5^{H43W}. PD-1 ligation induced K27-linked polyubiquitination and lysosomal degradation of γ_{c}, which were blocked in MARCH5-deficient cells. In contrast, PD-1 ligation had no marked effects on the protein level of γ_{c} in MARCH5-deficient cells reconstituted with ectopically-expressed wild-type MARCH5. These results suggest that PD-1 signal leads to MARCH5-mediated K27-linked polyubiquitination and degradation of γ_{c}, but does not affect MARCH5 activity per se. Mechanistically, the inventors found that PD-1 ligation induced the transcription factor BATF (basic leucine zipper ATF-like transcription factor), which transcriptionally induced MARCH5 by binding to the promoter of MARCH5 gene. BATF-deficiency impaired PD-L1-induced up-regulation of MARCH5 mRNA and down-regulation of γ_{c} protein level. Our experiments suggest that PD-1 signal induces the transcription factor BATF, which in turn induces expression of the E3 ubiquitin ligase MARCH5, leading to K27-linked polyubiquitination and lysosomal degradation of γ_{c}.

The experimental examples further identified USP5 as an enzyme that constitutively deubiquiting γ_{c}. USP5 removed K27-linked polyubiquitin moieties of γ_{c} conjugated by MARCH5. Knockout of USP5 increased PD-1 ligation-induced K27-linked polyubiquitination of γ_{c} and down-regulated its protein level in cells. These results suggest that USP5 acts as a constitutive guard for γ_{c} stability to ensure proper responses of CD8⁺ T and NK cells to the γ_{c} family cytokines. Consistently, the experiments showed that MARCH5-deficiency potentiated the γ_{c} family cytokine-triggered signaling and immune activation, whereas USP5-deficiency had the opposite effects. Taken together, the experiments suggest that the BATF-MARCH5-γ_{c} axis mediates PD-1-triggered inhibition of γ_{c} family cytokine-triggered signaling and immune activation.

The experimental examples also suggest that SHP2 mediates another mechanism responsible for PD-1-triggered inhibition of γ_{c} family cytokine-triggered signaling and immune activation. SHP2 is recruited to and activated in the complex of PD-1 upon PD-1 ligation by PD-L1, and the activated SHP2 mediates dephosphorylation of the TCR proximal kinases which leads to suppression of T cells. In the current experiments, it was found that PD-1 ligation promoted the association of γ_{c} with SHP2. Overexpression of gain-of-function SHP2 mutants (D61G or E76K) reduced JAK3-mediated phosphorylation of γ_{c}^{Y357}. Knockout of SHP2 impaired PD-1 ligation-induced γ_{c}^{Y357} dephosphorylation. Knockout of SHP2 also increased the γ_{c} family cytokine-induced phosphorylation of γ_{c}^{Y357} and STAT5^{Y694/Y699} Reconstitution of wild-type γ_{c} but not γ_{c}^{Y357F} in γ_{c}-deficient HPB-ALL cells restored IL-7- and IL-9-induced phosphorylation of STAT5^{Y694/Y699}. Taken together, these results suggest that PD-1 ligation-triggered SHP2 activation induces dephosphorylation of γ_{c}^{Y357}, resulting in desensitization of γ_{c}-mediated signaling and immune activation.

Based on our results, the instant inventors contemplate a model on the regulatory mechanisms of γ_{c} stability and activity by PD-1 signaling. In TME with high expression of PD-L1 in tumor cells, PD-1 signal in immune cells are hijacked and activated. The activated PD-1 recruits and activates SHP2, which subsequently mediates dephosphorylation of γ_{c}^{Y357F}, leading to its inactivation and unresponsiveness to γ_{c} family cytokines. On the other hand, PD-1 signal induces the transcription factor BATF, which induces expression of the membrane-associated E3 ubiquitin ligase MARCH5. MARCH5 is recruited to γ_{c} and mediates its K27-linked polyubiquitination at K315 and lysosomal degradation. Therefore, PD-1 signaling suppresses the γ_{c} family cytokine-triggered immune activation via two distinct mechanisms.

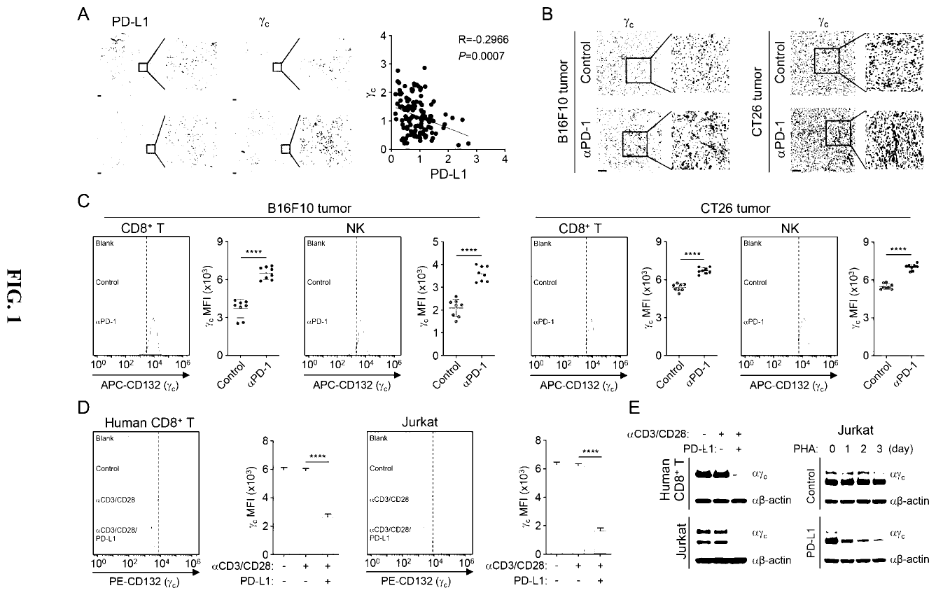

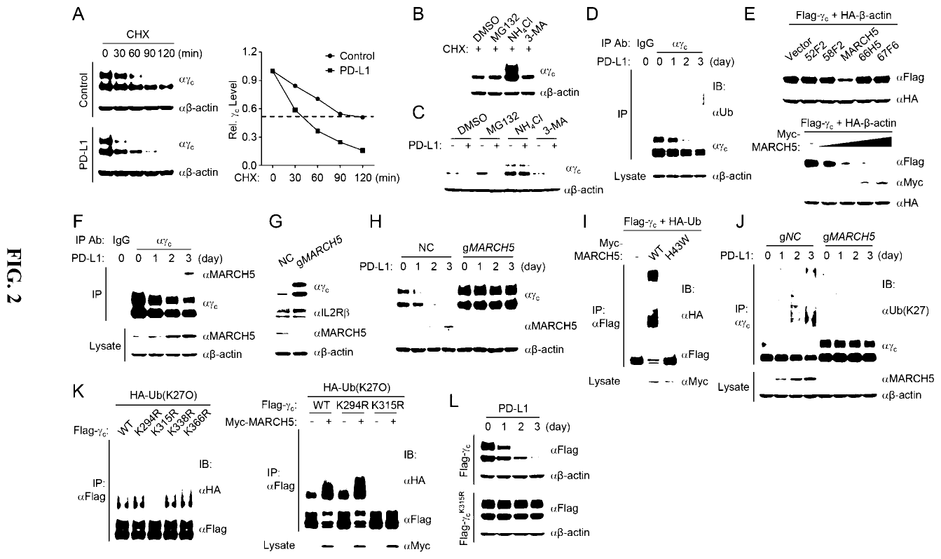

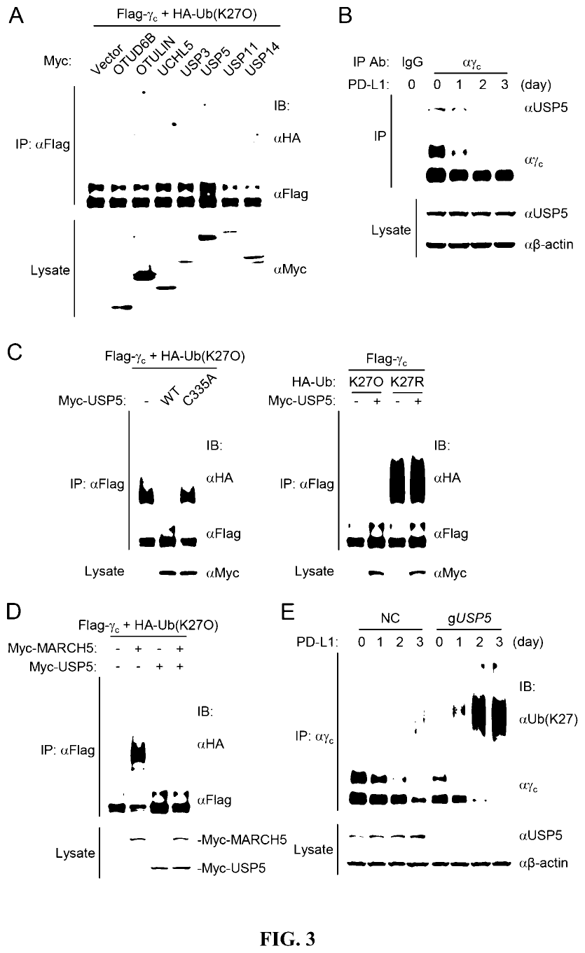

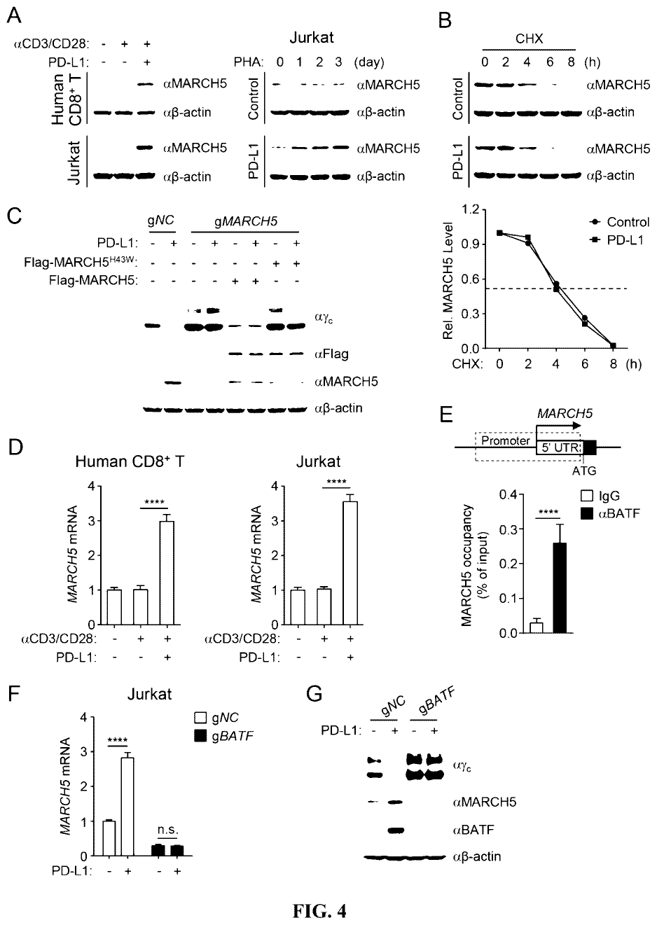

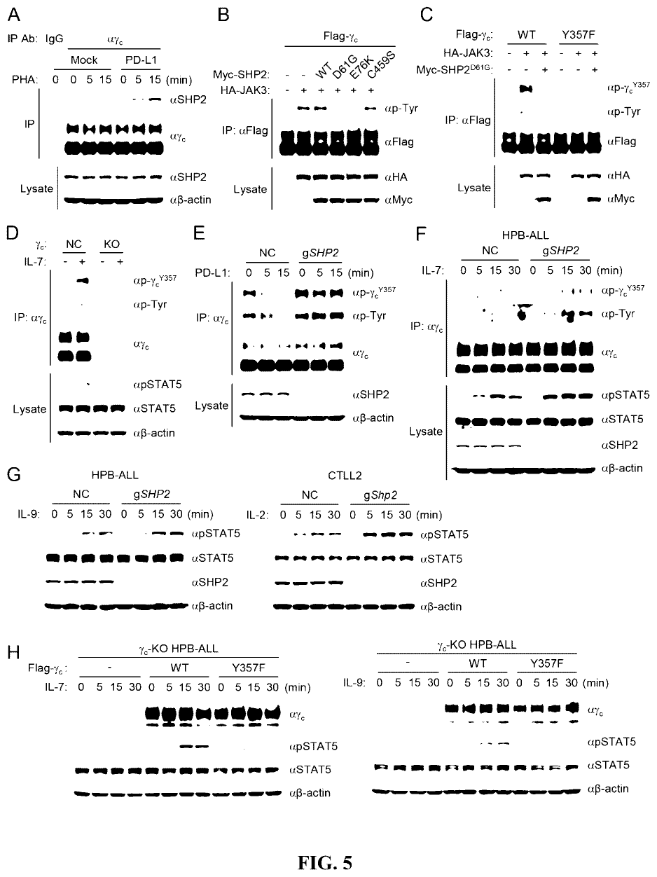

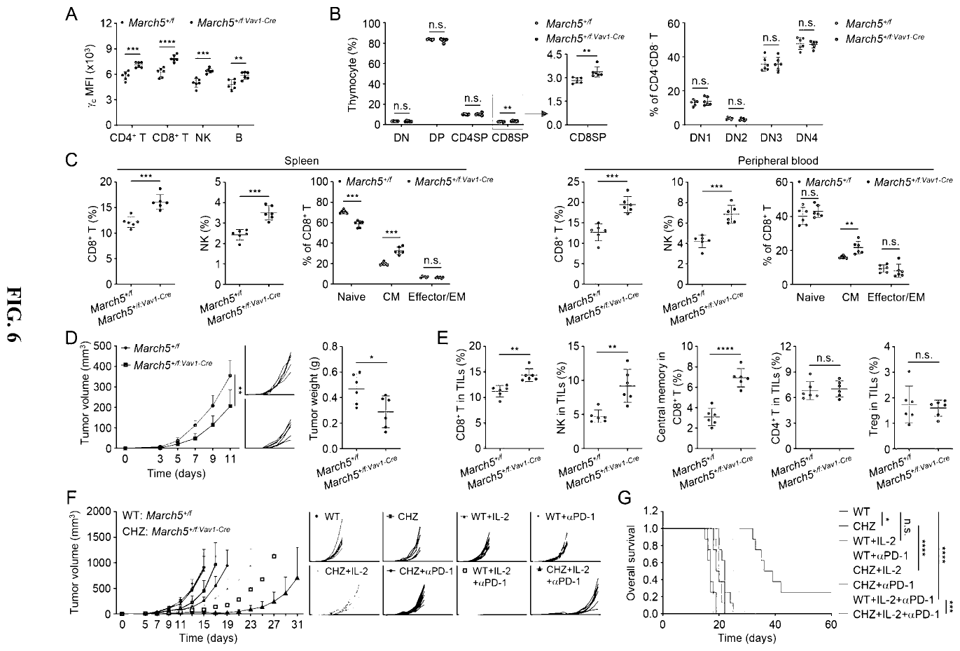

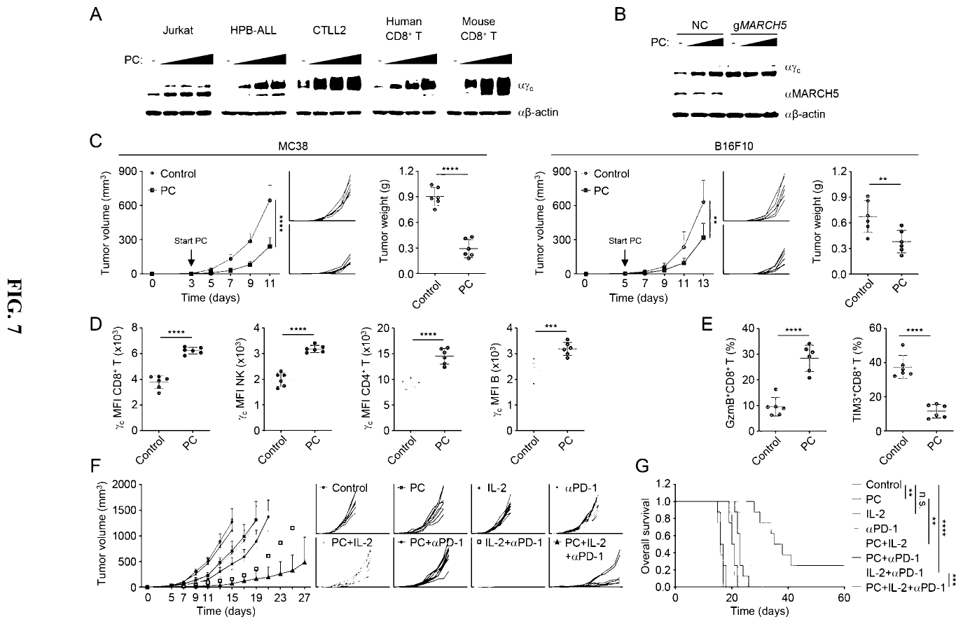

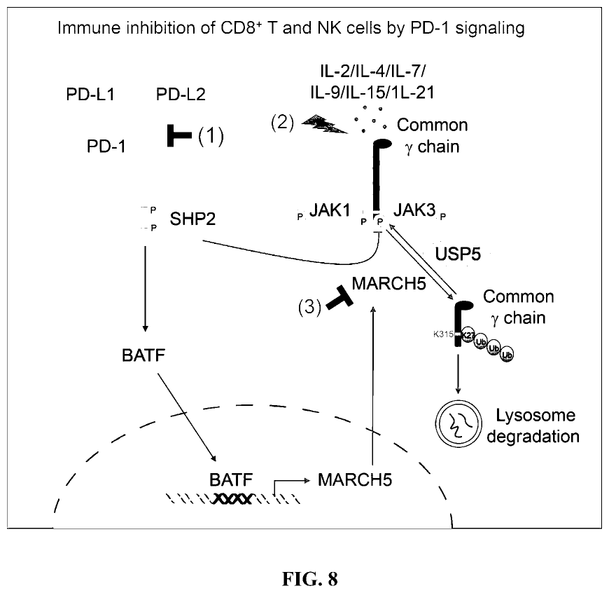

As shown in the experiments, PD-1-triggered SHP2 activation and dephosphorylation of γ_{c} occurred in minutes, whereas PD-1-triggered induction of MARCH5 and degradation of γ_{c} was obvious one day after PD-1 ligation. It is contemplated that PD-1 signal inhibits γ_{c} family cytokine-triggered immune activation via the two mechanisms in a temporal manner. Targeting of components involved in these regulatory mechanisms, such as inhibiting SHP2, inhibiting MARCH5 and/or activating USP5, can increase the efficacy of cancer immunotherapy by PD-1 blockade and the γ family cytokines (FIG. 8).

E3 ubiquitin-protein ligase MARCH5 is also known as membrane-associated ring finger (C3HC4) 5, MITOL or RNF153. A representative UniProt ID for the human MARCH5 protein is Q9NX47. A representative protein sequence is provided in NP_060294 and a representative mRNA sequence is provided in NM_017824. MARCH5 is localized in the mitochondrial outer membrane and has four transmembrane domains.

The instant inventors screened for inhibitors for MARCH5 among known compounds, and have identified pitavastatin (such as its calcium salt), lonafarnib, tucidinostat, belinostat, mocetinostat, pracinostat, risedronate, entinostat, chidamide, and vorinostat as having inhibiting activities. Their structures are listed in Table A below.

TABLE A
MARCH5 Inhibitors

| Inhibitor | Structure |
|---|---|
| Pitavastatin | 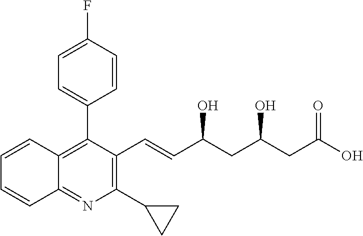 [SMILES: O=C(O)C[C@H](O)C[C@H](O)/C=C/c1c(C2CC2)nc2ccccc2c1-c1ccc(F)cc1] |
| Lonafarnib | 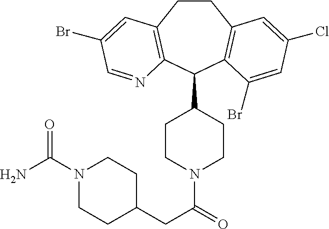 [SMILES: NC(=O)N1CCC(CC(=O)N2CCC([C@H]3c4ncc(Br)cc4CCc4cc(Cl)cc(Br)c43)CC2)CC1] |
| Tucidinostat | 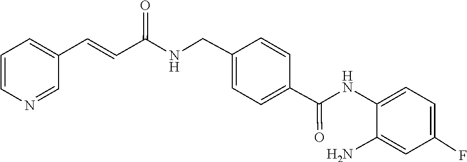 [SMILES: Nc1cc(F)ccc1NC(=O)c1ccc(CNC(=O)/C=C/c2cccnc2)cc1] |
| Belinostat | 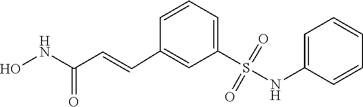 [SMILES: O=C(/C=C/c1cccc(S(=O)(=O)Nc2ccccc2)c1)NO] |
| Mocetinostat | 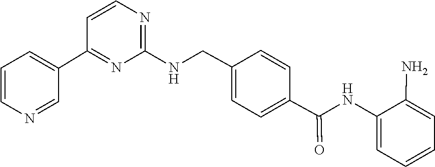 [SMILES: Nc1ccccc1NC(=O)c1ccc(CNc2nccc(-c3cccnc3)n2)cc1] |
| Pracinostat | 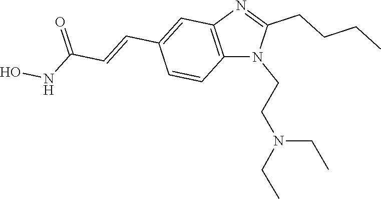 [SMILES: CCCCc1nc2cc(/C=C/C(=O)NO)ccc2n1CCN(CC)CC] |
| Risedronate | 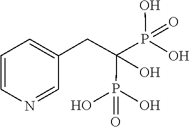 [SMILES: O=P(O)(O)C(O)(Cc1cccnc1)P(=O)(O)O] |
| Entinostat | 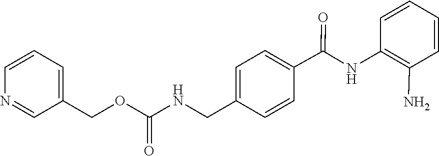 [SMILES: Nc1ccccc1NC(=O)c1ccc(CNC(=O)OCc2cccnc2)cc1] |
| Chidamide | 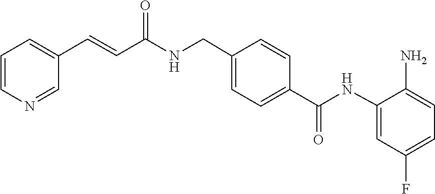 [SMILES: Nc1ccc(F)cc1NC(=O)c1ccc(CNC(=O)/C=C/c2cccnc2)cc1] |
| Vorinostat | 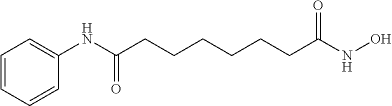 [SMILES: O=C(CCCCCCC(=O)Nc1ccccc1)NO] |

Pitavastatin calcium (PC) is a lipophilic statin and potent inhibitor of HMG-CoA reductase. Interestingly, some other HMG-CoA reductase inhibitors, such as lovastatin, simvastatin, fluvastatin sodium and rosuvastatin calcium, up-regulated the level of γ_{c}, suggesting that inhibition of HMG-CoA reductase does not have an effect on γ_{c} level. Therefore, pitavastatin-induced down-regulation of γ_{c} is independent of its inhibition of HMG-CoA reductase.

When a MARCH5 inhibitor, e.g., pitavastatin calcium (PC), was used in combination with PD-1 blockade and IL-2, the combination significantly improved the efficacy of anti-tumor immunotherapy in mice. By contrast, no such synergism was observed between rosuvastatin calcium and IL-2 on tumor suppression. The data showed that PC up-regulated the level of γ_{c} in a MARCH5-dependent manner. In MARCH5-deficient cells, the basal level of γ_{c} was increased and PC treatment did not further increase its protein level. PC treatment up-regulated the level of γ_{c} in tumor-infiltrating lymphocytes, inhibited tumor growth, and sensitized the anti-tumor effects of IL-2 as well as its combination with PD-1 blocking antibody. There results demonstrate that MARCH5 inhibitors can be utilized as potential drugs for combination immunotherapy of cancer.

In accordance with one embodiment of the present disclosure, provided is a method for treating cancer in a patient in need thereof. In some embodiments, the method entails administering to the patient an effective amount of an agent that inhibits the biological activity or expression of MARCH5 (membrane-associated ring finger 5) or SHP2 (src homology region 2 domain-containing phosphatase-2) or increases the biological activity or expression of USP5 (ubiquitin specific peptidase 5). In some embodiments, the method further includes administration of a common gamma-chain cytokine and/or a PD-1 inhibitor or a PD-L1 inhibitor.

Agents capable of inhibiting the biological activity or expression of a target protein can be readily obtained. One such example is an antibody or antigen-binding fragment. Types of antibodies and fragments are described in more detail above, and methods of obtaining antibodies and fragments are known in the art. In one embodiment, the agent is an anti-MARCH5 antibody or antigen-binding fragment. In one embodiment, the agent is an anti-SHP2 antibody or antigen-binding fragment.

Another example of such agents is an inhibitory RNA. An inhibitory RNA is an RNA molecule that can inhibit gene expression at the post-transcriptional level. There are several types of inhibitory RNA, as further set forth below.

MicroRNAs (miRNAs): miRNAs are small non-coding RNAs that regulate gene expression by targeting specific mRNAs for degradation or translational repression. miRNAs are transcribed from DNA and then processed by the cell into mature miRNAs that can recognize and bind to complementary sequences on target mRNAs, leading to their degradation or translational repression.

Small interfering RNAs (siRNAs): siRNAs are another type of small non-coding RNA that can induce gene silencing by targeting specific mRNAs for degradation or translational repression. siRNAs are typically introduced into cells by transfection or viral transduction and can be used for research or therapeutic purposes.

Short hairpin RNAs (shRNAs): shRNAs are RNA molecules that can induce gene silencing by mimicking the structure of a miRNA precursor. They are typically introduced into cells by transfection or viral transduction and can be used for research or therapeutic purposes.

Piwi-interacting RNAs (piRNAs): piRNAs are a type of small non-coding RNA that play a role in regulating transposons and maintaining genomic stability in germ cells. piRNAs interact with a class of proteins known as Piwi proteins and can induce gene silencing by epigenetic mechanisms, such as DNA methylation or histone modification.

Anti-sense RNAs (asRNAs): asRNAs are RNA molecules that are complementary to specific mRNAs and can induce gene silencing by hybridizing to the mRNA and preventing its translation or promoting its degradation.

An agent that inhibits the biological activity or expression of a target protein, such as MARCH5, can also be a small molecule. Such small molecules can be identified readily from library screening, as exemplified in the experimental examples. Without limitation, a small molecule that inhibits MARCH5 may be pitavastatin, lonafarnib, tucidinostat, belinostat, mocetinostat, pracinostat, risedronate, entinostat, chidamide, vorinostat, or a salt thereof. In one embodiment, a small molecule that inhibits MARCH5 is pitavastatin or pitavastatin calcium.

Agents that can increase the biological activity or expression can also be readily obtained. In one example, the agent is a recombinant version of the target protein. Alternatively, a polynucleotide, such as DNA or mRNA, that encodes the target protein can also be used. In another example, the endogenous gene of the target protein may be engineered to increase expression. Without limitation, in one embodiment, the agent is a recombinant USP5 protein or polynucleotide encoding the USP5 protein.

In some embodiments, the patient has been treated with a common gamma-chain cytokine, is undergoing a treatment with a common gamma-chain cytokine, or is prescribed to receive a treatment with a common gamma-chain cytokine.

In some embodiments, the patient has been treated with a PD-1 inhibitor or a PD-L1 inhibitor, is undergoing a treatment with a PD-1 inhibitor or a PD-L1 inhibitor, or is prescribed to receive a treatment with a PD-1 inhibitor or a PD-L1 inhibitor.

In some embodiments, the treatment entails administration of both an agent of the instant disclosure and a common gamma-chain cytokine and a PD-1 inhibitor or a PD-L1 inhibitor. In some embodiments, the treatment entails administration of all of (a) an agent of the instant disclosure, (b) a common gamma-chain cytokine and (c) a PD-1 inhibitor or a PD-L1 inhibitor.

Examples of common gamma-chain cytokines and PD-1 inhibitor or a PD-L1 inhibitors are provided in more detail below.

Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, sarcoma, skin cancer, squamous cell carcinoma, bone cancer, melanoma, renal cell carcinoma, and kidney cancer.

In some embodiments, the tumors being treated are those that are particularly challenging to treat with conventional immuno-oncological therapies, such as with antibodies targeting immune checkpoints (ICPs). Sometimes, such tumors are referred to as “cold tumors” or “nonimmunogenic tumors.” In some embodiments, accordingly, the present disclosure provides methods and uses for treating cold tumors with multi-specific antibodies disclosed herein.

In some embodiments, a nonimmunogenic tumor is one that is not infiltrated with T cells, or that is deficient in T cell filtration, in antigen presenting cells (APCs), or in T cell activation, or has deficit in T cell homing into the tumor bed. All of prostate cancer, pancreatic cancer, and leukemia are nonimmunogenic. The vast majority of breast cancer (95%), colorectal cancer (95%), gastric cancer (87%), head and neck cancer (84%), liver cancer (83%), esophageal cancer (86%), cervical cancer (87%), and thyroid cancer (87%) are also nonimmunogenic. In addition, 83% of lung cancer, 79% of bladder cancer, 77% of kidney cancer, 70% uterus cancer, and 66% melanoma are also nonimmunogenic.

Identification of nonimmunogenic, or cold tumors can also be made with measurements of type, density and location of immune cells within the tumors. For instance, Galon and Bruni (Nature Reviews Drug Discovery volume 18, pages 197-218 (2019)) describes a standardized scoring system, Immunoscore, based on the quantification of two lymphocyte populations (CD3 and CD8), e.g., in resected tissues, for guided stratification of hot and cold tumors. The Immunoscore ranges from Immunoscore 0 (I0, for low densities, such as absence of both cell types in both regions) to I4 (high immune cell densities in both locations). By classifying cancers according to their immune infiltration, the scoring system provides an immune-based classification of tumors, including a definition of “hot” (highly infiltrated, Immunoscore I4) and “cold” (non-infiltrated, Immunoscore I0) tumors.

In some embodiments, the tumor is resistant to a treatment with immune checkpoint inhibitors, such as PD-L1 inhibitors, PD-1 inhibitors, CTLA-4 inhibitors, or the combinations thereof. In some embodiments, the cancer is prostate cancer, pancreatic cancer, or leukemia. In some embodiments, the cancer is breast cancer, colorectal cancer, gastric cancer, head and neck cancer, liver cancer, esophageal cancer, cervical cancer, or thyroid cancer. In some embodiments, the cancer is lung cancer, bladder cancer, kidney cancer, uterus cancer, or melanoma.

Additional diseases or conditions associated with increased cell survival, that may be treated, prevented, diagnosed and/or prognosed with the agents of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

A specific dosage and treatment regimen for any particular patient will depend upon a variety of factors, including the particular agents used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the agents include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the agents of the disclosure into the central nervous system by any suitable route, including intraventricular and intrathecal injection; intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent.

It may be desirable to administer the agents or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an agent of the disclosure, care must be taken to use materials to which the protein does not absorb.

### PD-1 Inhibitors

A PD-1 inhibitor is a molecule that binds to and inhibits the biological activity of the PD-1 protein. Programmed cell death protein 1, also known as PD-1 and CD279 (cluster of differentiation 279), is a protein on the surface of cells that has a role in regulating the immune system's response to the cells of the human body by down-regulating the immune system and promoting self-tolerance by suppressing T cell inflammatory activity. Examples are anti-PD-1 antibodies and fragments thereof, such as those described below.

Pembrolizumab (formerly MK-3475 or lambrolizumab, Keytruda) is an anti-PD-1 monoclonal antibody developed by Merck and first approved by the Food and Drug Administration in 2014 for the treatment of melanoma. It was later approved for metastatic non-small cell lung cancer and head and neck squamous cell carcinoma.

Nivolumab (Opdivo) is an anti-PD-1 monoclonal antibody developed by Bristol-Myers Squibb and first approved by the FDA in 2014 for the treatment of melanoma. It was later approved for squamous cell lung cancer, renal cell carcinoma, and Hodgkin's lymphoma.

Cemiplimab (Libtayo) is an anti-PD-1 monoclonal antibody developed by Regeneron Pharmaceuticals and first approved by the FDA in 2018 for the treatment of cutaneous squamous cell carcinoma (CSCC) or locally advanced CSCC who are not candidates for curative surgery or curative radiation.

Spartalizumab (PDR001) is an anti-PD-1 monoclonal antibody developed by Novartis to treat both solid tumors and lymphomas.

Camrelizumab (SHR1210) is an anti-PD-1 monoclonal antibody introduced by Jiangsu HengRui Medicine Co., Ltd. that recently received conditional approval in China for the treatment of relapsed or refractory classical Hodgkin lymphoma.

Sintilimab (IB1I308) is an anti-PD-1 monoclonal antibody developed by Innovent and Eli Lilly for patients with non-small cell lung cancer (NSCLC).

Tislelizumab (BGB-A317) is a humanized IgG4 anti-PD-1 monoclonal antibody developed by BeiGene for solid tumors and hematologic cancers.

Dostarlimab (TSR-042, WBP-285) is a humanized monoclonal antibody against PD-1 under investigation by GlaxoSmithKline.

INCMGA00012 (MGA012) is a humanized IgG4 monoclonal antibody developed by Incyte and MacroGenics.

AMP-224 is an anti-PD-1 monoclonal antibody by AstraZeneca/MedImmune and GlaxoSmithKline.

AMP-514 (MEDI0680) is an anti-PD-1 monoclonal antibody by AstraZeneca.

### PD-L1 Inhibitors

A PD-L1 inhibitor is a molecule that binds to and inhibits the biological activity of the PD-L1 protein. Programmed death-ligand 1 (PD-L1) also known as cluster of differentiation 274 (CD274) or B7 homolog 1 (B7-H1) is a protein that in humans is encoded by the CD274 gene. Examples are anti-PD-L1 antibodies and fragments thereof, such as those described below.

Atezolizumab (Tecentriq) is a humanized anti-PD-L1 IgG1 antibody developed by Roche Genentech. It has been approved by the FDA for urothelial carcinoma and non-small cell lung cancer.

Avelumab (Bavencio) is a human anti-PD-L1 IgG1 antibody developed by Merck Serono and Pfizer. Avelumab has been approved by the FDA for the treatment of metastatic merkel-cell carcinoma.

Durvalumab (Imfinzi) is a human anti-PD-L1 IgG1 antibody developed by AstraZeneca. Durvalumab has been approved by the FDA for the treatment of urothelial carcinoma and unresectable non-small cell lung cancer after chemoradiation.

KN035 is an anti-PD-L1 antibody with subcutaneous formulation currently under clinical evaluations in the US, China, and Japan.

CK-301 is an anti-PD-L1 antibody being developed by Checkpoint Therapeutics.

Some small peptide and small molecule inhibitors are also being developed. Examples are shown below.

AUNP12 is a 29-mer peptide as the first peptic PD-1/PD-L1 inhibitor developed by Aurigene and Laboratoires Pierre Fabre that is being evaluated in clinical trial, following promising in vitro results.[28]

CA-170, discovered by Aurigene/Curis as the PD-L1 and VISTA antagonist, was indicted as a potent small molecule inhibitor in vitro. The compound is currently under phase I clinical trial over mesothelioma patients.

BMS-986189 is a macrocyclic peptide discovered by Bristol-Myers Squibb of which the pharmacokinetics, safety and tolerability is currently being studied on healthy subjects.

### Common Gamma-Chain Cytokines

The common gamma chain (γ_{c}) (or CD132), also known as interleukin-2 receptor subunit gamma or IL-2RG, is a cytokine receptor sub-unit that is common to the receptor complexes for interleukin receptors such as interleukin-2 (IL-2), IL-4, IL-7, IL-9, IL-15 and IL-21.

Conversely, the common gamma chain (γ_{c}) cytokine family include IL-2, IL-4, IL-7, IL-9, IL-15 and IL-21, named after the γ_{c} subunit (CD132) shared by these cytokines. Common gamma-chain cytokine signals provide pro-survival cues, inhibit pro-apoptotic signals, upregulate metabolic activities and promote expression of select transcription factors, which determine lineage fate and maturation of lymphocyte subsets.

The term common gamma-chain cytokine also encompasses biological equivalents of these cytokines. The biological functions and their structural domains of these cytokines are well known. Conservative amino acid substitutions at residues not critical to the function of a cytokine can be made without substantively impacting the function. Proteins with such substitutions can be considered their biological equivalents, or simply variants.

In some embodiments, a common gamma-chain cytokine a recombination cytokine protein. In some embodiments, a common gamma-chain cytokine is provided as a polynucleotide encoding the cytokine.

In some embodiments, a common gamma-chain cytokine can be combined with an anti-PD-1 or anti-PD-L1 antibody or fragment to form a fusion protein.

### MARCH5 Inhibition for the Treatment of Other Diseases

The inhibition of MARCH5 is also contemplated to be able to treat diseases and conditions besides cancers. In one embodiment, it is contemplated that MARCH5 inhibitors can be effective at improving a subject's innate immunity. Accordingly, a MARCH5 inhibitor of the instant disclosure can be used to treat infection.

Infection is the invasion of an organism's body tissues by disease-causing agents, their multiplication, and the reaction of host tissues to these organisms and the toxins they produce. An infection can be caused by infectious agents such as viruses, viroids, prions, bacteria, nematodes such as parasitic roundworms and pinworms, arthropods such as ticks, mites, fleas, and lice, fungi such as ringworm, and other macroparasites such as tapeworms and other helminths. In one aspect, the infectious agent is a bacterium, such as Gram negative bacterium. In one aspect, the infectious agent is virus, such as DNA viruses, RNA viruses, and reverse transcribing viruses. Non-limiting examples of viruses include Adenovirus, Coxsackievirus, Epstein-Barr virus, Hepatitis A virus, Hepatitis B virus, Hepatitis C virus, Herpes simplex virus, type 1, Herpes simplex virus, type 2, Cytomegalovirus, Human herpesvirus, type 8, HIV, Influenza virus, Measles virus, Mumps virus, Human papillomavirus, Parainfluenza virus, Poliovirus, Rabies virus, Respiratory syncytial virus, Rubella virus, Varicella-zoster virus.

It has been observed that knockdown of endogenous MARCH5 significantly inhibits FUNDC1 degradation and enhances mitochondrial sensitivity toward mitophagy-inducing stresses. It is contemplated that MARCH5 inhibition can prevent, treat or delay the progression of Parkinson's disease.

In some embodiments, the MARCH5 inhibitor is one or more of pitavastatin (such as its calcium salt), lonafarnib, tucidinostat, belinostat, mocetinostat, pracinostat, risedronate, entinostat, chidamide, and vorinostat. In some embodiments, the MARCH5 inhibitor is an antibody or an inhibitory RNA, without limitation.

### Pharmaceutical Compositions and Combinations

As provided, a pharmaceutical composition is described that includes an agent that inhibits the biological activity or expression of MARCH5 (membrane-associated ring finger 5) or SHP2 (src homology region 2 domain-containing phosphatase-2) or increases the biological activity or expression of USP5 (ubiquitin specific peptidase 5). In some embodiments, the composition further includes a common gamma-chain cytokine and/or a PD-1 inhibitor or a PD-L1 inhibitor. The combinations may be provided as a kit or package.

Such a composition may be suitable for oral, parenteral, topical administration or for administration by inhalation. Accordingly, a pharmaceutical composition comprising at least agent according to the present disclosure may be administered parenterally, such as intravenously, or intramuscularly, or subcutaneously. Alternatively, agent may be administered via a non-parenteral route, such as per-orally or topically. In a preferred embodiment, a pharmaceutical composition comprising agent according to the present disclosure is administered intravenously or subcutaneously.

In an embodiment, the present disclosure provides a pharmaceutical composition comprising one or more agents according to the present disclosure for use in the prevention and/or treatment of a disease. In an embodiment, the present disclosure provides a pharmaceutical composition comprising one or more agents according to the present disclosure for the use as a medicament. In an embodiment, the present disclosure provides a pharmaceutical composition comprising one or more agents according to the present disclosure for use in the prevention and/or treatment of an autoimmune disease and/or inflammatory disease and/or cancer.

The agents here may be combined into a single composition for use. Nevertheless, in some embodiments, they are administered separately to the same patient.

### EXAMPLES

### Example 1: PD-1 Signal Negatively Regulates Stability and Activity of the Common Cytokine Receptor γ Chain to Suppress Anti-Tumor Immunity

This example discovered that PD-1 signal transcriptionally induced the E3 ubiquitin ligase MARCH5, which targeted γ_{c} for K27-linked polyubiquitination and lysosomal degradation. Additionally, PD-1 signal activated SHP2, which mediated dephosphorylation of γ_{c} and inhibition of signaling triggered by the γ_{c} family cytokines. These results suggest that PD-1 signal antagonizes immune activation triggered by the γ_{c} family of cytokines through two distinct mechanisms. Consistently, PD-1 blockade restored the response of tumor-infiltrating CD8⁺ T and NK cells to γ_{c} family cytokines. In addition, a MARCH5 inhibitor potently improved the efficacy of immunotherapy triggered by PD-1 blockade and IL-2 in mouse tumor models. Our results reveal the mechanisms on how PD-1 signal inhibits γ_{c} family cytokine-triggered activation of CD8⁺ T and NK cells and provides potential targets for increased efficacy of combination immunotherapy of cancer.

### Methods

### Reagents and Antibodies

Reagents and antibodies used in this study were purchased from the indicated companies: recombinant human PD-L1-Fc fusion protein (BPS Bioscience), anti-human CD3E (clone OKT3, Biolegend), anti-human CD28 (clone CD28.2, Biolegend), PHA (Sigma), polybrene (Millipore), SYBR (Bio-Rad), cycloheximide (Sigma), MG132 (Sigma), NH₄Cl (Sigma), 3-MA (Sigma), Pitavastatin calcium (Aladdin), human IL-2 (SL Pharm), human IL-7 (Peprotech), and human IL-9 (Peprotech). The antibody that specifically recognizes phosphorylated Y357 of γ_{c} was raised by immunizing rabbits with a synthetic peptide of human γ_{c} (₃₅₄HSP(Y-p)WAPPC₃₆₂) by ABclonal Technology (Wuhan).

### Cells

Jurkat cells were obtained from American Type Culture Collection. HEK293 cells were originally provided by the National Jewish Health (Denver, CO). HPB-ALL cells were provided by Wuhan university. CTLL2 cells were obtained from Cell Resource Center (IBMS, CAMS/PUMC). Human CD8⁺ T cells were obtained from Milestone® Biotechnologies. Mouse CD8⁺ T cells were isolated from the spleen of 6-8-weeks-old C57BL/6 mice by positive selection magnetic beads (STEM CELL Technologies). B16F10 cells, CT26 cells and MC38 cells were provided by Wuhan University.

Jurkat cells were cultured in RPMI 1640 (GIBCO) supplemented with 10% FBS (Biological Industries) and 1% penicillin-streptomycin (Thermo Fisher Scientific). HPB-ALL cells were cultured in RPMI 1640 supplemented with 10% FBS, 1% penicillin-streptomycin, 1% non-essential amino acids (GIBCO), 2 mM L-glutamine (GIBCO), 1 mM sodium pyruvate (GIBCO) and 55 μM β-mercaptoethanol (Sigma-Aldrich). CTLL2 cells were cultured in RPMI 1640 supplemented with 10% FBS, 1% penicillin-streptomycin and IL-2 (100 IU/ml). Human CD8⁺ T cells and mouse CD8⁺ T cells were cultured in RPMI 1640 supplemented with 10% FBS, 1% penicillin-streptomycin, 1% non-essential amino acids, 2 mM L-glutamine, 1 mM sodium pyruvate, 55 μM β-mercaptoethanol and IL-2 (400 IU/ml). HEK293, B16F10, CT26 and MC38 cells were cultured in DMEM (GIBCO) supplemented with 10% FBS and 1% penicillin-streptomycin. All cells were detected negative for mycoplasma.

### Constructs

Mammalian expression plasmids for Flag-, HA-, or Myc-tagged γ_{c}, MARCH5, β-actin, IL2RB, IL4R, IL7R, OTUD6B, OTULIN, UCHL5, USP3, USP5, USPl1, USP14, SHP2, JAK3 and their mutants, as well as pSuper. Retro-shRNA plasmids for MARCH5 were constructed by standard molecular biology techniques. Guide-RNA plasmids targeting γ_{c}, MARCH5, USP5, BATF and SHP2 were constructed into a lentiCRISPR V2 vector, which was provided by Wuhan University.

### Transfection

HEK293 cells were transfected by standard calcium phosphate precipitation. The empty control plasmid was added to ensure that each transfection receives the same amount of total DNA.

### CRISPR-Cas9 Knockout

Double-stranded oligonucleotides corresponding to the target sequences were cloned into the Lenti-CRISPR-V2 vector, which were co-transfected with the packaging plasmids into HEK293 cells. Two days after transfection, the viruses were harvested, ultra-filtrated (0.45 μm filter, Millipore) and used to infect cells in the presence of polybrene (8 μg/mL). The infected cells were selected with puromycin (Jurkat: 1 μg/mL, HPB-ALL: 2 μg/mL, CTLL2: 4 μg/mL) for at least 6 days.

### Co-Immunoprecipitation, Ubiquitination and Immunoblotting Analysis

Cells were lysed in 1 ml of NP-40 lysis buffer (20 mM Tris-HCl, 150 mM NaCl, 1 mM EDTA, 10% Nonidet P-40, 1l % Triton X-100, 10 g/ml aprotinin, 10 μg/ml leupeptin, and 1 mM phenylmethylsulfonyl fluoride, PMSF). For each immunoprecipitation reaction, a 0.4 ml aliquot of lysate was incubated with 0.5-2 μg of the indicated antibody or control IgG and 35 μL of a 1:1 slurry of Protein-G Sepharose (GE Healthcare) at 4° C. for 3 h. The Sepharose beads were washed three times with 1 ml of lysis buffer containing 500 mM NaCl. The precipitates were fractionated by SDS-PAGE, and immunoblotting analysis was performed with the indicated antibodies. For ubiquitination assays, the immunoprecipitants were re-extracted in NP-40 lysis buffer containing 1% SDS and denatured by heating for 10 min. The supernatants were diluted with regular lysis buffer until the concentration of SDS was decreased to 0.1%, following by re-immunoprecipitation with the indicated antibodies. The immunoprecipitants were analyzed by immunoblotting with the ubiquitin antibody.

qPCR

Total RNA was isolated for qPCR analysis to measure mRNA abundance of the indicated genes. Data shown are the relative abundance of the indicated mRNAs normalized to that of GAPDH. The qPCR data was collected with Bio-Rad CFX96 (Version 3.1) and analyzed with Bio-Rad CFX Manager (Version 3.1).

### Chromatin Immunoprecipitation (ChIP)

ChIP was performed according to the manufacture's instruction (Sigma). Ten million cells were fixed with 1% formaldehyde for 10 min, quenched with 0.125 M glycine for 5 min at 37° C. and lysed in SDS Lysis Buffer. Cell lysate was sonicated by Bioruptor Pico Sonifier to shear chromatin DNA to a size range of 200-1000 bp. The supernatant was diluted 10-fold in ChIP Dilution Buffer and precleared with 60 mL agarose beads for 30 min. The supernatant fraction was immunoprecipitated with the indicated antibodies (2 μg) against BATF overnight at 4° C. The antibody-chromatin complexes were pulled down with protein A agarose/salmon sperm DNA beads (Sigma) for 1 h at 4° C.

### Flow Cytometry Analysis

Cells were subjected to stain with the indicated antibodies for 30 min in 4° C. The cells were analyzed and data were acquired with BD Fortessa X-20 and FACSDiva 7 software following the exemplified gating strategy for flow cytometry analysis. The data were processed using FlowJo software.

### Mass Spectrometry

Jurkat cells (1×10⁸) were used for mass spectrometry analysis. Endogenous 7_{c} was immunoprecipitated with anti-γ_{c} and desalted, then analyzed by mass spectrometry. Mass spectrometry analysis was performed as previously described by SpecAlly (Wuhan) Life Science and Technology Company.

### Human NSCLC Samples

The human NSCLC samples were provided by Wuhan university. All cases were re-reviewed by pathologists from the Department of Pathology of Tongji Hospital for the confirmation of tumor histology and tumor content. All cases were used in this study were performed with written patient informed consents and approved by the Institutional Review Committee of Tongji Hospital, Tongji Medical College, Huazhong University of Science and Technology, and the Medical Ethic Committee of the School of Medicine, Wuhan University.

### Immunohistochemistry (IHC) Staining

IHC staining was performed as previously described. In brief, the slides were deparaffinized in xylene, and rehydrated sequentially in 100%, 95%, and 75% ethanol for 5 min. The antigen retrieval was performed by heating slides in a microwave for 30 min in sodium citrate buffer (pH 6.0) or 0.5 mm EDTA buffer (pH 8.0). The sections were cooled down naturally to room temperature and quenched in 3% hydrogen peroxide to block endogenous peroxidase activity. The sections were incubated with antibodies overnight at 4° C. Next, a secondary biotinylated immunoglobulin G antibody solution and an avidin-biotin peroxidase reagent were added onto slides. After washing with phosphate buffer saline, 3,3′-diaminobenzidine tetrachloride was added to the sections, followed by counterstaining with hematoxylin (Beyotime Biotech). Signals were imaged with an Aperio VERSA 8 (Leica) multifunctional scanner and quantified with the software Image-Pro Plus 6.0.

### MARCH5 Conditional Knockout Mice and Genotyping

March5^{flox/flox} mice were generated by the Animal Center of Wuhan University Medical Research Institute. Amplification of the WT allele with primers F and R generates a 1020-bp fragment, whereas amplification of the disrupted allele with primers F and R generates a 1088-bp fragment. To generate MARCH5 hematopoietic-specific knockout mice, March5^{flox/flox} mice were bred to Vav1-Cre mice to generate March5^{f/f:Vav1-Cre} mice. Genotyping of the Vav1-Cre mice by PCR was performed using the following primers which produces a 205 bp fragment. All animal utility was carried out in compliance with the Institutional Animal Care and Use Committee (TACUC) guidelines and approved by the Animal Care and Ethics Committee of Wuhan University Medical Research Institute.

### In Vivo Experimental Therapy in Syngeneic Mouse Tumor Models

Age- and sex-matched March5^{+/f} and March5^{+/f:Vav1-Cre} mice, C57BL/6j or Balb/c mice (all age of 6-8 weeks) were anaesthetized and subcutaneously injected with the indicated mouse tumor cells (5×10⁵ in 200 μL PBS). The mice were euthanized when the tumor size was bigger than 15 mm of the mean tumor diameter or tumor volume reaches 2000 mm³ or deemed as died.

Anti-PD-1 therapy in C57BL/6j and Balb/c mice: mice were intraperitoneally injected with control or anti-PD-1 (BE0273, 100 μg per mouse, dissolved in PBS, BioXCell) every three days (four times in total) five days after B16F10 (C57BL/6j mice) or CT26 (Balb/c mice) cells inoculation. Tumor-bearing mice were euthanized on day 17. Tumor tissues were analyzed by IHC staining and TILs were analyzed by flow cytometry.

The MC38 and B16F10 tumor models in March5^{+/f} and March5^{+/f:Vav1-Cre} mice: on the day of three after tumor cells implantation, tumor sizes were measured every two days by caliper. Tumour-bearing mice were euthanized on day 13. TILs were analyzed by flow cytometry.

Pitavastatin calcium (PC) therapy in C57BL/6j mice: mice were intraperitoneally injected with control or PC (P129617, 5 mg/kg/day, dissolved in PBS, Aladdin) three (MC38 tumor) or five (B16F10 tumor) days after inoculation of tumor cells, and tumor sizes were measured every two days by caliper. Tumor-bearing mice were euthanized on day 13 (MC38 tumor) or day 15 (B16F10 tumor). TILs were analyzed by flow cytometry.

IL-2 and anti-PD-1 therapy in March5^{+/f} and March5^{+/f:Vav1-Cre} mice: mice were intraperitoneally injected with control, IL-2 (50000 IU per mouse, dissolved in PBS) or anti-PD-1 (100 μg per mouse, dissolved in PBS) five days after inoculation of MC38 cells. Tumor size and mouse survival were measured every two days from day 5.

PC, IL-2 and anti-PD-1 therapy in C57BL/6j mice: mice were intraperitoneally injected with control, PC (5 mg/kg, dissolved in PBS), IL-2 (50000 IU per mouse, dissolved in PBS) and anti-PD-1 (100 μg per mouse, dissolved in PBS) five days after inoculation of MC38 cells. Tumor size and mouse survival were measured every two days from day 5.

For survival studies, mice were sacrificed when the tumor size was bigger than 15 mm of the mean tumor diameter, tumor volume exceeded 2000 mm³, or tumor had ulcers with a diameter reached 10 mm. Statistical analysis was performed using the GraphPad Prism 8 software. Kaplan-Meier survival curves and corresponding log-rank (Mantel-Cox) tests were used to evaluate the statistical differences between groups in survival studies. There is a significant difference when the P<0.05.

### Isolation of Tumor Infiltrated Immune Cells

Tumor tissues were separated from the mice and cut into pieces. The tumor tissues were suspended with 2 ml of tumor digestion buffer (1×HBSS buffer with 5 mg/ml collagenase II and 0.1% DNaseI) and rotated for at 37° C. for 1 h. The cell suspension was filtered using a 70-μm filter to obtain single-cell suspension. The lymphocytes were isolated by density-gradient centrifugation using 40% and 70% Percoll (GE). The TILs were stained using fluorescently labelled antibodies for different markers. Cells were analyzed and data were acquired with BD Fortessa X-20 and FACSDiva 7 software following the exemplified gating strategy for flow cytometry analysis. The data were processed using FlowJo software.

### Statistics and Reproducibility

Data were analyzed using a student's unpaired t-test, multiple t-test or two-way ANOVA with GraphPad Prism 8. The correlation study was analyzed using a Spearman rank correlation test. The number of asterisks represents the degree of significance with respect to P values, with the latter presented within each FIG. or FIG. legend. All the biochemical experiments, particularly immunoblotting analysis, were repeated for at least two times with similar results. The reproducibility of other experiments is described in the respective FIG. legends.

### Results

### PD-1 Signals Down-Regulation of γ_{c} Level

To explore whether there is a regulatory relationship between PD-1 signal and the γ_{c} family cytokines, we firstly analyzed the correlation between PD-L1 and γ_{c} level in TME. Immunohistochemistry (IHC) staining showed that γ_{c} level was negatively correlated with PD-L1 level in human non-small cell lung cancer (NSCLC) biopsies (FIG. 1A). Next, we used a PD-1 antibody to disrupt the PD-1/PD-L1 signaling axis in mouse B16F10 and CT26 tumor models and then analyzed γ_{c} level in the tumor tissues. The results showed that PD-1 blockade up-regulated γ_{c} level in tumor tissues (FIG. 1B). Tumor-infiltrating CD8⁺ T and NK cells are the main killer cells, and both of them rely on the γ_{c} family cytokines for proliferation and activation. Therefore, we analyzed γ_{c} levels in tumor-infiltrating CD8⁺ T and NK cells. The results indicated that the levels of γ_{c} were increased in these cells after PD-1 blockade treatment (FIG. 1C), suggesting that PD-1 signal negatively regulates γ_{c} level in tumor-infiltrating CD8⁺ T and NK cells.

We next investigated whether PD-1 signal regulates γ_{c} level in human T cells. Flow cytometry analysis indicated that γ_{c} level was not markedly changed after TCR activation by anti-CD3 and anti-CD28 antibodies, but was significantly down-regulated after PD-1 ligation by PD-L1 in both primary human CD8⁺ T cells and Jurkat T cells (FIG. 1D). Immunoblotting analysis further confirmed that PD-L1 but not PHA (which activates TCR) treatment caused down-regulation of γ_{c} level in both primary human CD8⁺ T cells and Jurkat T cells (FIG. 1E). These results suggest that PD-1 signal down-regulates γ_{c} level in human T cells.

### MARCH5 Mediates K27-Linked Polyubiquitination and Degradation of γ_{c}

We next investigated the mechanisms responsible for PD-1 signal-triggered down-regulation of γ_{c}. qPCR experiments indicated that PD-L1 stimulation did not affect the mRNA level of γ_{c} in human primary CD8⁺ T cells or Jurkat cells, suggesting that PD-1 signal regulates γ_{c} at the protein but not mRNA level. The protein synthesis inhibitor cycloheximide (CHX) treatment showed that γ_{c} had a shorter half-life after PD-L1 stimulation in Jurkat cells (FIG. 2A), suggesting that PD-1 ligation promotes γ_{c} degradation. γ_{c} degradation after termination of protein synthesis by CHX was markedly inhibited by the lysosomal inhibitor ammonium chloride (NH₄Cl) but not the proteasome inhibitor MG132 or autophagosome inhibitor 3-methyladenine (3-MA) in Jurkat cells (FIG. 2B). PD-L1-induced degradation of γ_{c} was also inhibited by NH₄Cl but not MG132 or 3-MA in Jurkat cells (FIG. 2C). Together, these results suggest that PD-1 signaling results in γ_{c} degradation by the lysosomal route.

Next, we found that PD-L1 induced polyubiquitination and down-regulation of γ_{c} in Jurkat cells, which was most dramatic at 2 days after PD-L1 stimulation (FIG. 2D). We next attempted to identify the E3 ubiquitin ligases that catalyze degradation of γ_{c}. We screened 196 ubiquitin-related proteins for their abilities to regulate γ_{c} level by co-transfection experiments in HEK293 cells. These efforts led to the identification of MARCH5, which caused down-regulation of γ_{c} in a dose-dependent manner (FIG. 2E) but had no marked effects on the γ_{c} family cytokine receptor IL2RB, IL4R or IL7R. Endogenous co-immunoprecipitation experiments indicated that γ_{c} was basally associated with MARCH5 and PD-L1 stimulation promoted their association in PD-1-expressing Jurkat cells (FIG. 2F).

Interestingly, these experiments also indicated that MARCH5 was induced after PD-L1 stimulation, which reached to the highest level at 3 days after PD-L1 stimulation, and the induction of MARCH5 was correlated with down-regulation of γ_{c} (FIG. 2F). We generated MARCH5-deficient Jurkat cells using the CRISPR-Cas9 method and found that MARCH5-deficiency up-regulated the level of γ_{c} but not IL2RP (FIG. 2G). In addition, MARCH5-deficiency increased γ_{c} level in human T lymphoid leukemia HPB-ALL cells and murine T lymphocyte CTLL2 cells. Consistently, MARCH5-deficiency increased the γ_{c} family cytokine IL-7- and IL-9-induced phosphorylation of STAT5^{Y694/Y699} in HPB-ALL cells or IL-2-induced phosphorylation of STAT5^{Y694/Y699} in CTLL2 cells, which is a hallmark of STAT5 activation. In addition, NH₄Cl treatment inhibited MARCH5-mediated degradation of γ_{c} in HEK293 cells. These data suggest that MARCH5 mediates lysosomal degradation of γ_{c} and inhibition of the γ_{c} family cytokine-triggered signaling in various cells.

We next investigated the functions of the E3 ubiquitin ligase MARCH5 in γ_{c} degradation triggered by PD-1 signaling. We found that MARCH5-deficiency impaired PD-L1-induced γ_{c} degradation in PD-1-expressing Jurkat cells (FIG. 2H), suggesting that MARCH5 mediates PD-L1-induced γ_{c} degradation. Overexpression of MARCH5 but not its inactive mutant MARCH5^{H43W} promoted polyubiquitination of γ_{c} in HEK293 cells (FIG. 2I). Utilizing ubiquitin mutants in which one or six lysine residues are replaced with arginine (R), we found that MARCH5 increased K27—but not other lysine residue-linked polyubiquitination of γ_{c}. Endogenous ubiquitination assays indicated that PD-L1 stimulation increased K27-linked polyubiquitination and degradation of γ_{c}, and these effects were impaired in MARCH5-deficient PD-1-expressing Jurkat cells (FIG. 2J). Collectively, these results suggest that MARCH5 mediates K27-linked polyubiquitination and degradation of γ_{c} following PD-1 ligation.

To further identify the residues in γ_{c} that are conjugated with K27-linked polyubiquitin chains by MARCH5, we individually mutated each of the 4 lysine residues within the intracellular region (aa284-369) of γ_{c}, K294, K315, K338 and K363, to arginine and examined whether these mutants could be modified by K27-linked polyubiquitination. The results indicated that mutation of K315 but not the other 3 lysine residues in γ_{c} to arginine dramatically reduced its K27-linked polyubiquitination, and MARCH5 increased K27-linked polyubiquitination and down-regulation of wild-type γ_{c} and γ_{c}^{K294R} but not γ_{c}^{K315R} (FIG. 2K). Consistently, PD-L1 stimulation induced down-regulation of wild-type γ_{c} but not γ_{c}^{K315R} (FIG. 2L). Reconstitution of yC^{K315R} in γ_{c}-deficient HPB-ALL cells increased IL-7- and IL-9-induced phosphorylation of STAT5 694^{Y699} in comparison to cells reconstituted with wild-type γ_{c}. In these reconstitution experiments, the mRNA levels of γ_{c}^{K315R} and wild-type γ_{c} were comparable, but the protein level of γ_{c}^{K315R} was dramatically up-regulated compared to its wild-type counterparts. Taken together, these results suggest that MARCH5 targets γ_{c}^{K315} for its K27-linked polyubiquitination and degradation.

### USP5 Antagonizes MARCH5-Mediated γ_{c} Polyubiquitination and Degradation

We next attempted to identify deubiquitinate enzymes that are responsible for removing K27-linked polyubiquitin moieties conjugated to γ_{c}. γ_{c}-bound proteins were immunoprecipitated with anti-ye and analyzed by mass spectrometry. Among the 184 proteins identified, 7 are deubiquitinate enzymes. Co-transfection experiments indicated that only USP5 but not the other 6 enzymes removed K27-linked polyubiquitin moieties from γ_{c} in HEK293 cells (FIG. 3A). Endogenous co-immunoprecipitation experiments indicated that γ_{c} was constitutively associated with USP5 in Jurkat cells (FIG. 3B). USP5 but not its enzymatic inactive mutant USP5^{C335A} removed K27-linked polyubiquitin moieties from γ_{c} and up-regulated the level of γ_{c} in HEK293 cells (FIG. 3C). In addition, USP5 removed K27-linked polyubiquitination of γ_{c} catalyzed by MARCH5 (FIG. 3D). Endogenous ubiquitination assays indicated that PD-L1 stimulation induced K27-linked polyubiquitination of γ_{c}, which was increased in USP5-deficient Jurkat cells (FIG. 3E). In these experiments, the protein level of γ_{c} in USP5-deficient cells was also dramatically down-regulated compared with those in wild-type cells (FIG. 3E). USP5-deficiency also down-regulated the level of γ_{c} in HPB-ALL and CTLL2 cells, and inhibited IL-2-, IL-7- and IL-9-induced phosphorylation of STAT5 at Y694/Y699 in these cells. These data suggest that USP5 positively regulates γ_{c} level as well as the γ_{c} family cytokine-triggered signaling in various cells.

### PD-1 Signal Promotes γ_{c} Degradation by Inducing MARCH5 Transcription

In our experiments, we found that PD-1 ligation had no marked effects on the protein level of USP5 as well as its association with γ_{c} (FIG. 3B). USP5-deficiency down-regulated γ_{c} level but did not affect PD-L1-induced γ_{c} degradation (FIG. 3E). These results suggest that USP5 is not required for PD-1 ligation-induced γ_{c} degradation. On the other hand, we found that the protein level of MARCH5 was not markedly changed after TCR activation by PHA, but was up-regulated following PD-L1 stimulation (FIG. 4A, also see FIG. 2F, H&J). PD-L1 stimulation did not affect the half-life of MARCH5 in CHX-treated cells (FIG. 4B), suggesting that PD-1 ligation does not affect its stability. MARCH5-deficiency caused up-regulation of γ_{c} protein level, which was reversed by reconstitution with wild-type MARCH5 but not MARCH5^{H43W1} in Jurkat cells. Additionally, PD-1 ligation down-regulated γ_{c} level in wild-type but not MARCH5-deficient cells ectopically reconstituted with wild-type MARCH5 in Jurkat cells (FIG. 4C). These results suggest that PD-1 ligation regulates MARCH5 level at mRNA but not protein level.

qPCR analysis indicated that PD-L1 treatment up-regulated MARCH5 mRNA level in human CD8⁺ T and Jurkat cells (FIG. 4D). It has been demonstrated that PD-1 ligation induces expression of certain transcription factors including the AP-1 family member BATF, which forms a complex with IRF4 and JunB to induce transcription of downstream genes, leading to impairment of T cell proliferation and cytokine production. Analysis of the Gene Transcription Regulation Database (GTRD, gtrd.biouml.org) indicates that BATF/IRF4/JunB complex can bind to the promoter region of MARCH5 gene. Consistently, chromatin immunoprecipitation (ChIP) experiments confirmed that BATF bound to the promoter region of MARCH5 gene in Jurkat cells (FIG. 4E). BATF-deficiency caused down-regulation and impairment of PD-L1-induced up-regulation of MARCH5 mRNA level in Jurkat cells (FIG. 4F). BATF-deficiency also up-regulated γ_{c} level and inhibited PD-L1-induced down-regulation of γ_{c} level (FIG. 4G). These results suggest that PD-1 signal transcriptionally induces MARCH5 in a BATF-dependent manner.

PD-1-Triggered SHP2 Activation Induces Dephosphorylation of γ_{c}^{Y357}

PD-1 ligation triggers clustering of PD-1 with TCR and recruitment of the allosteric dephosphorylating enzyme SHP2, which is activated and mediates dephosphorylation of TCR proximal signaling molecules such as LCK and ZAP70, leading to inactivation of T cells. Our biochemical purification and mass spectrometry analysis indicated that SHP2 was a potential ye-bound proteins. Endogenous co-immunoprecipitation experiments indicated that γ_{c} was weakly associated with SHP2 and PD-L1 stimulation promoted their association at 5 minutes post stimulation (FIG. 5A). These results suggest that PD-1 ligation promotes a physical association of SHP2 with γ_{c}.

We next investigated whether SHP2 mediates dephosphorylation of γ_{c}. It has been demonstrated that the tyrosine kinase JAK3 mediates phosphorylation of γ_{c} upon stimulation of the γ_{c} family cytokines. Overexpression of the SHP2 gain-of-function mutant D61A or E76K but bot the wild-type SHP2 or dominant negative SHP2 mutant C459S abolished JAK3-mediated phosphorylation of γ_{c} (FIG. 5B). There are 4 tyrosine residues in the intracellular region (aa284-369) of γ_{c}. Mutagenesis indicated that mutation of Y357 but not the other 3 tyrosine residues to phenylalanine impaired JAK3-mediated tyrosine phosphorylation of γ_{c}. Sequence analysis indicated that γ_{c}^{Y357} was conserved in various vertebrate species. To determine whether SHP2 dephosphorylates γ_{c}^{Y357}, we generated a rabbit polyclonal antibody specific for Y357-phosphorylated γ_{c} (p-γ_{c}^{Y357}). Immunoblotting analysis confirmed that γ_{c}^{Y357} was phosphorylated following co-expression of JAK3, which was reversed by the gain-of-function mutant SHP2^{D61G} in HEK293 cells (FIG. 5C). Endogenous γ_{c}^{Y357} was phosphorylated following IL-7 stimulation, which was not seen in un-stimulated or γ_{c}-deficient HPB-ALL cells (FIG. 5D).

Most importantly, PD-L1 stimulation caused dephosphorylation of γ_{c}^{Y357} which was dramatic as early as 5 minutes post stimulation, while SHP2-deficiency abolished the effects of PD-L1-induced dephosphorylation of γ_{c}^{Y357} in Jurkat cells (FIG. 5E). SHP2-deficiency also enhanced IL-7-induced phosphorylation of γ_{c}^{Y357} (FIG. 5F) as well as IL-2-, IL-7- and IL-9-induced phosphorylation of STAT5^{Y694/Y699} (FIG. 5F&G). Reconstitution of wild-type γ_{c} but not γ_{c}^{K357F} in γ_{c}-deficient HPB-ALL cells restored IL-7- and IL-9-induced phosphorylation of STAT5^{Y694/Y699} (FIG. 5H). These results suggest that PD-1 ligation triggers SHP2 activation to desensitize γ_{c}-mediated signaling.

### MARCH5-Deficiency Improves Anti-Tumor Immunity and Suppresses Tumor Growth

It has been reported that March5 homozygous deletion is embryonically lethal. To investigate the physiological functions of MARCH5, March5^{flox/flox} mice were generated and crossed with Vav1-Cre mice to obtain March5 hematopoietic-specific knockout strain (March5^{+/f:Vav1-Cre}). Unexpectedly, the March5^{+/f:Vav1-Cre} mice were born normally, but all died during 4-6 weeks after birth. Thus, we used March5^{+/f} and March5^{+/f:Vav1-Cre} mice for further investigation. We verified that the mRNA and protein levels of MARCH5 in the bone marrow (BM), spleen and thymus of March5^{+/f:Vav1-Cre} mice were about half to that in March5^{+/f} mice. Flow cytometry analysis indicated that MARCH5-deficiency up-regulated the level of γ_{c} in CD4⁺ T, CD8⁺ T, NK and B cells from the spleen (FIG. 6A). MARCH5-deficiency had no marked effects on the percentages of double-negative (DN), double-positive (DP), CD4⁺ single-positive (CD4SP) cells, but increased the percentage of CD8⁺ single-positive (CD8SP) cells in the thymus (FIG. 6B). March5^{+/f:Vav1-Cre} mice exhibited significantly higher percentages of CD8⁺ T and NK cells and slightly lower percentages of B and CD4⁺ T cells in spleen and the peripheral blood (FIG. 6C). The percentage of CD44^{high}CD62L^{high} central memory (CM) CD8⁺ T cells were also increased in total CD8⁺ T cells of spleen and the peripheral blood of March5^{+/f:Vav1-Cre} mice (FIG. 6C). The percentage of CD44^{high}CD62L^{high} central memory CD4⁺ T cells did not show significant difference in CD4⁺ T cells of spleen and the peripheral blood of March5^{+/f:Vav1-Cre} mice. These results suggest that MARCH5 negatively regulates γ_{c} level as well as CD8⁺ T and NK cell development in mice.

We next used mouse MC38 colorectal carcinoma and B16F10 melanoma models to investigate the biological functions of MARCH5 in anti-tumor immunity. In these models, March5^{+/f:Vav1-Cre} mice showed slower tumor progression than March5^{+/f} mice (FIG. 6D). We isolated tumor-infiltrating lymphocytes (TILs) from the tumor tissues of March5^{+/f} and March5^{+/f:Vav1-Cre} mice and analyzed the immunological changes in tumors. The results showed that March5^{+/f:Vav1-Cre} mice exhibited significantly higher percentages of CD8⁺ T, NK and central memory CD8⁺ T cells in TILs comparing to March5^{+/f} mice (FIG. 6E), while the percentages of CD4⁺ T, Treg and B cells in TILs were similar between March5^{+/f} and March5^{+/f:Vav1-Cre} mice. To determine whether MARCH5-deficiency affects the activation of tumor-infiltrating CD8⁺ T cells or the profile of exhausted T cells, we examined the T cell activation maker Granzyme B (GzmB) and the exhausted T cell marker TIM3 on infiltrated CD8⁺ T cells in the mice tumor models. The results showed that MARCH5-deficiency down-regulated the percentage of TIM3⁺ cells in CD8⁺ T cells, suggesting MARCH5-deficiency inhibited the exhaustion of CD8⁺ T cells. These results suggest that MARCH5 negatively regulates anti-tumor immunity in mouse models.

Based on their powerful abilities to stimulate proliferation of cytotoxic CD8⁺ T and NK cells, certain γ_{c} family cytokines such as IL-2 have long been used in clinical cancer immunotherapy. Since MARCH5-deficiency leads to higher level of γ_{c} in CD8⁺ T and NK cells, we reasoned that this would improve the efficacy of the γ_{c} family cytokines in anti-tumor immunotherapy. Consistently, administration of IL-2 in March5^{+/f:Vav1-Cre} mice showed increased efficacy in suppression of tumor growth and improvement of the overall survival comparing to that in March5^{+/f} mice (FIG. 6F&G). We further combined IL-2 with PD-1 blocking antibody for tumor immunotherapy in March5^{+/f} and March5^{+/f:Vav1-Cre} mice. The results showed that the anti-tumor effects of combined IL-2 with PD-1 blockade in March5^{+/f:Vav1-Cre} mice were superior to that in March5^{+/f} mice, which eliminated tumors in 2 out of 8 March5^{+/f:Vav1-Cre} mice (FIG. 6F&G). Taken together, these results suggest that MARCH5-deficiency sensitizes the anti-tumor effects of IL-2 as well as its combination with PD-1 blocking antibody.

Combination of a MARCH5 Inhibitor with IL-2 and PD-1 Blockade Significantly Increases Antitumor Efficacy

Our results reveal an important role of MARCH5 in PD-1-triggered immune suppression, which suggest that MARCH5 is a potential target for cancer immunotherapy. Unfortunately, no pharmacological inhibitors of MARCH5 have been reported. We designed a reporter system in which HEK293 cells are co-transfected with two plasmids encoding MARCH5 and γ_{c}-luciferase fusion protein respectively. In this system, inhibition of MARCH5 by an inhibitor would result in increased luciferase activity in reporter assays.

Using this system, we screened a collection of Food and Drug Administration-approved drugs and identified 10 compounds that increased luciferase activity to more than 1.5-folds (Table 1). Further confirmative experiments identified Pitavastatin calcium (PC) as the most potent inhibitor that specifically increased the luciferase activity in MARCH5-expressing but not control HEK293 cells. Pitavastatin is a unique lipophilic statin and potent inhibitor of HMG-CoA reductase with a strong effect on lowering plasma total cholesterol and triacylglycerol. Pitavastatin has also been reported to have pleiotropic beneficial effects such as suppression of inflammation, regulation of angiogenesis and osteogenesis, improvement of endothelial function and arterial stiffness. Consistent with a specific inhibition of MARCH5 activity in the reporter system, PC treatment up-regulated the level of γ_{c} in a dose-dependent manner in all examined cells including primary human and mouse CD8⁺ T cells, and Jurkat, HPB-ALL and CTLL2 cells (FIG. 7A).

Statin drugs, such as lovastatin, simvastatin, fluvastatin sodium and rosuvastatin calcium, are thought to target HMG-CoA reductase. Therefore, we examined the effects of these statin drugs on the γ_{c} level. The results indicated that these examined statin drugs up-regulated γ_{c} level, suggesting that inhibition of the HMG-CoA reductase does not have an effect on the γ_{c} level. In MARCH5-deficient cells, the basal level of γ_{c} was increased and PC treatment did not further increase its protein level (FIG. 7B). There results suggest that PC is a specific inhibitor of MARCH5 and capable of inhibiting MARCH5-mediated γ_{c} degradation.

We further tested the anti-tumor effects of PC in mouse MC38 colorectal carcinoma and B16F10 melanoma models. Administration of PC alone significantly suppressed tumor growth in mice (FIG. 7C). Administration of PC also resulted in higher level of γ_{c} in tumor-infiltrating lymphocytes (FIG. 7D). The percentages of CD8⁺ T, NK, CD4⁺ T, Treg and B cells in tumor-infiltrating lymphocytes did not show significant difference after PC administration. PC treatment up-regulated the percentage of GzmB⁺ cells and reduced the percentage of TIM3⁺ cells in infiltrated CD8⁺ T cells, suggesting that PC enhances infiltration of CD8⁺ cytotoxic T cells and inhibits exhaustion of CD8⁺ T cells (FIG. 7E). We further investigated the anti-tumor effects of PC combined with IL-2 and PD-1 blockade. Consistently, PC significantly increased the efficacies of IL-2 or IL-2 plus PD-1 blocking antibody on tumor suppression (FIG. 7F) and overall survival of mice (FIG. 7G). In addition, we investigated whether the other HMG-CoA reductase inhibitors such as Rosuvastatin Calcium affect the antitumor efficacies of IL-2. Administration of PC or rosuvastatin calcium alone suppressed tumor growth in mice. However, only PC but not rosuvastatin calcium has a synergistic effect with IL-2 on tumor suppression, suggesting that PC promotes antitumor immunity via its inhibition of MARCH5 but not HMG-CoA reductase. These results suggest that administration of MARCH5 inhibitor leads to increased efficacies of cancer immunotherapy by IL-2 plus PD-1 blockade.

TABLE 1
Compounds Inhibiting MARCH5

| Rank | Drug | Rel. Luc. Act | Target |
|---|---|---|---|
| 1 | Lonafarnib | 2.88 | Raf/Ras |
| 2 | Pitavastatin calcium | 2.06 | HMG-CoA reductase |
| 3 | Tucidinostat | 1.86 | HDAC1/2/3/10 |
| 4 | Belinostat | 1.85 | HDAC |
| 5 | Mocetinostat | 1.83 | HDAC1/2/3/11 |
| 6 | Pracinostat | 1.75 | HDAC |
| 7 | Risedronate Sodium | 1.60 | HBV |
| 8 | Entinostat | 1.60 | HDAC1/3 |
| 9 | Chidamide | 1.59 | HDAC1/2/3/8 |
| 10 | Vorinostat | 1.58 | HDAC1/2/3/6/8 |

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A method for treating cancer or infection in a patient in need thereof, comprising administering to the patient an effective amount of an agent that inhibits the biological activity or expression of MARCH5 (membrane-associated ring finger 5) or SHP2 (src homology region 2 domain-containing phosphatase-2) or increases the biological activity or expression of USP5 (ubiquitin specific peptidase 5).

2. The method of claim 1, further comprising administering to the patient a common gamma-chain cytokine, or wherein the patient has received or is prescribed to receive a therapy comprising a common gamma-chain cytokine.

3. The method of claim 2, wherein the common gamma-chain cytokine is selected from the group consisting of IL-2, IL-4, IL-7, IL-9, IL-15, IL-21, and combinations thereof.

4. The method of claim 2, wherein the common gamma-chain cytokine comprises IL-2.

5. The method of claim 1, further comprising administering to the patient a PD-1 inhibitor or a PD-L1 inhibitor, or wherein the patient has received or is prescribed to receive a therapy comprising a PD-1 inhibitor or a PD-L1 inhibitor.

6. The method of claim 5, wherein the PD-1 or PD-L1 inhibitor is an anti-PD-1 or anti-PD-L1 antibody of antigen-binding fragment thereof.

7. The method of claim 5, wherein the PD-1 inhibitor is selected from the group consisting of pembrolizumab, nivolumab, cemiplimab, spartalizumab, camrelizumab, sintilimab, tislelizumab, dostarlimab, INCMGA00012, AMP-224 and AMP-514.

8. The method of claim 5, wherein the PD-L1 inhibitor is selected from the group consisting of atezolizumab, avelumab, durvalumab, KN035, CK-301, AUNP12, CA-170, BMS-986189, B6 and B12-01.

9. The method of claim 6, wherein the anti-PD-1 or anti-PD-L1 antibody is a fusion protein that further comprises a common gamma-chain cytokine.

10. The method of claim 1, wherein the agent is a small molecule MARCH5 inhibitor.

11. The method of claim 10, wherein the small molecule MARCH5 inhibitor is selected from the group consisting of pitavastatin, lonafarnib, tucidinostat, belinostat, mocetinostat, pracinostat, risedronate, entinostat, chidamide, vorinostat, and salts thereof.

12. The method of claim 10, wherein the agent is pitavastatin calcium.

13. The method of claim 1, wherein the agent is an antibody or antigen-binding thereof that targets MARCH5 or SHP2.

14. The method of claim 1, wherein the agent is an inhibitory RNA that targets MARCH5 or SHP2.

15. (canceled)

16. The method of claim 1, wherein the agent is a recombinant USP5 protein or polynucleotide encoding the USP5 protein.

17. The method of claim 1, wherein the cancer is selected from the group consisting of bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, sarcoma, skin cancer, squamous cell carcinoma, bone cancer, melanoma, renal cell carcinoma, and kidney cancer.

18. The method of claim 1, wherein the cancer is a cold tumor.

19. (canceled)

20. A method for preventing or treating Parkinson's disease in a patient in need thereof, comprising administering to the patient an effective amount of an agent that inhibits the biological activity or expression of MARCH5 (membrane-associated ring finger 5).

21. The method of claim 20, wherein the agent is selected from the group consisting of pitavastatin, lonafarnib, tucidinostat, belinostat, mocetinostat, pracinostat, risedronate, entinostat, chidamide, vorinostat, and salts thereof.

22. A kit or package comprising (a) an agent that inhibits the biological activity or expression of MARCH5 (membrane-associated ring finger 5) or SHP2 (src homology region 2 domain-containing phosphatase-2) or increases the biological activity or expression of USP5 (ubiquitin specific peptidase 5), and (b1) a common gamma-chain cytokine and/or (b2) a PD-1 inhibitor or a PD-L1 inhibitor.
